(19) United States
(12) Patent Application Publication
(10) Pub. No.: US 2020/0347352 A1
(43) Pub. Date: Nov. 5, 2020

(54) METHODS AND SYSTEM OF HUMAN HEMOGENIC REPROGRAMING

(71) Applicant: Icahn School of Medicine at Mount Sinai, New York, NY (US)

(72) Inventors: Kateri A. Moore, New York, NY (US); Ihor R. Lemischka, New York, NY (US); Michael G. Daniel, New York, NY (US)

(21) Appl. No.: 16/964,739

(22) PCT Filed: Jan. 25, 2019
(86) PCT No.: PCT/US19/15117

§ 371 Date: Jul. 24, 2020

Related U.S. Application Data
(60) Provisional application No. 62/657,032, filed on Apr. 13, 2018.
(60) Provisional application No. 62/621,655, filed on Jan. 25, 2018.

(51) Int. Cl. C12N 5/0789; C12N 5/10; G01N 33/50; C12N 5/0775; A01N 1/02; A61K 35/28

(52) U.S. Cl. CPC C12N 5/0647; C12N 5/10; G01N 33/5044; A61K 2035/124; A01N 1/0221; A61K 35/28; G01N 33/5014; C12N 5/0667

(57) ABSTRACT

This disclosure provides a method for programming human somatic cells into hematopoietic stem cells (HSCs). The method includes inducing expression of the 3GF reprogramming transcription factor cocktail, including GATA2, GFI1B, GFI1, and FOS transcription factors, in human somatic cells. Further, this disclosure also demonstrates co-culturing HSCs with AFT024 stroma cells results in more functional cells, both qualitatively and quantitatively.

### CROSS-REFERENCE TO RELATED APPLICATIONS

This application claims priority under 35 U.S.C. § 119(e) to United States Provisional Patent Application No. 62/621,655, filed Jan. 25, 2018, and U.S. Provisional Patent Application No. 62/657,032, filed Apr. 13, 2018, the disclosures of which are incorporated herein by reference in their entireties.

### STATEMENT REGARDING FEDERALLY-SPONSORED RESEARCH

This invention was made with government support under Grant No. 1R01HL119404 awarded by the National Institutes of Health (NIH). The government has certain rights in the invention.

### FIELD OF THE INVENTION

This disclosure relates generally to methods and systems of hemogenic reprogramming and more specifically to programming human somatic cells into hematopoietic stem cells by inducing expression of specific transcription factors.

### BACKGROUND OF THE INVENTION

Hematopoietic stem cells (HSCs) generate all the cellular elements of the blood in a hierarchical manner, though work in this field proposes several alterations to the composition of the classical hierarchy. Multiple studies demonstrate an endothelial origin for multipotent HSCs, notably showing their emergence from a specific subset of endothelium called hemogenic endothelium (HE) which gives rise to HSCs via a process of cell budding termed endothelial-to-hematopoietic transition (EHT). Due to their ability to repopulate the entire hematopoietic system upon transplantation in both mice and humans, HSCs represent the currently established standard for stem cell therapy.

The source material required for these applications, however, remains in limited supply although several studies exist trying to expand these cells ex vivo or generate them de novo. This issue hinders the use of these cells for various in vitro applications, such as drug testing platforms and disease modeling systems. Additionally, HSCs notoriously die or differentiate in culture ex vivo (Clark, B.R., et al. (1997). Methods in molecular biology 75, 249-256). Allogeneic transplants, however, carry multiple risks of graft-versus-host disease and graft rejection due to poor HLA matching and a lack of ethnic diversity for sufficient matching material.

A paradigm shift in stem cell biology occurred when Takahashi and colleagues demonstrated that overexpression of a defined set of transcription factors (TFs) could reprogram differentiated somatic cells to iPSCs (Takahashi, K. et al. (2006). Cell 126, 663-676; Takahashi, K., et al. (2007). Cell 131, 861-872). Studies have been carried out to translate this idea of forced TF overexpression altering cell identity to the field of hematopoiesis, with multiple studies using different starting mouse or human cell populations, TF cocktails, or culture conditions to obtain various types of in vitro derived blood products de novo. Although the grand majority of these studies each contribute to the growing understanding of hematopoiesis, they fail to generate a bona fide HSC.

Thus, there exists a pressing need in the art for programming human somatic cells into HSCs.

### SUMMARY OF THE INVENTION

The disclosure addresses this need by providing a method for programming a human somatic cell into a hematopoietic stem cell. The method includes introducing into the human somatic cell a combination of transcription factors, wherein the combination comprises GATA binding protein 2 (GATA2), growth factor independent 1B (GFI1B), growth factor independent 1 (GFI1), and FBJ osteosarcoma oncogene (FOS).

The nucleic acid may be introduced by viral transduction, for example, by including one of gag, pol, and env coding sequences in the nucleic acids encoding the transcription factors. In some embodiments, the method may further include co-culturing the hematopoietic stem cell with a stromal cell, for example, an AFT024 stromal cell.

The human somatic cell may include, without limitation, fibroblasts, epithelial cells, bone marrow cells, differentiated hematopoietic cells, macrophages, hematopoietic progenitor cells, and peripheral blood mononuclear cells.

In one aspect, this disclosure provides a method for screening the cell for expression of a hemogenic endothelial cell marker or a hematopoietic stem cell marker. Examples of the hemogenic endothelial cell marker or the hematopoietic stem cell marker may include, without limitation, CD31, CD34, CD38^{lo/-}, CD41, CD43, CD45, CD49f, Thy1/CD90, CD105, CD117/c-kit, CD133, CD143, CD150, CD201, Sca-1, Tie2, VE-Cadherin, KDR/FLK1, Flk-2/Flt3, and CXCR4. In some embodiments, the hematopoietic stem cell marker is CD34 or CD49f. Also within the scope of this disclosure is a method for isolating the cell expressing a hemogenic endothelial cell marker or a hematopoietic stem cell marker.

In another aspect, this disclosure provides isolated hematopoietic stem cells obtained by the methods described above and a composition comprising isolated hematopoietic stem cells. The composition may additionally include a cryo-protectant.

In another aspect, this disclosure provides blood, cellular and acellular blood components, blood products or hematopoietic stem cells comprising the isolated hematopoietic cells described above.

In another aspect, this disclosure also provides a method of engraftment or cell replacement for autologous or non-autologous transplantation in a subject in need thereof comprising transferring to the subject the isolated hematopoietic cells described above.

In another aspect, this disclosure also provides a method for treating a subject who suffers from a condition or a disease that would benefit from hematopoietic stem cell transplantation. The method includes administering to the subject a therapeutically effective amount of the isolated hematopoietic stem cells described above. The condition or disease may include cancer, a congenital disorder, and vascular disease. In some embodiments, the isolated hematopoietic stem cell is autologous to the subject in need thereof.

In another aspect, this disclosure also provides a method for treating a subject who suffers from a condition or a disease that would benefit from hematopoietic stem cell transplantation, comprising administering to the subject a therapeutically effective amount of the isolated hematopoietic stem cells described above. The condition or disease may include multiple myeloma, leukemia, congenital neutropenia with defective stem cells, aplastic anemia, myelodysplastic syndrome, neuroblastoma, lymphoma, Ewing's Sarcoma, Desmoplastic small round cell tumor, chronic granulomatous disease, non-Hodgkin's lymphoma, Hodgkin's disease, acute myeloid leukemia, neuroblastoma, germ cell tumors, systemic lupus erythematosus (SLE), systemic sclerosis, amyloidosis, acute lymphoblastic leukemia, chronic myeloid leukemia, chronic lymphocytic leukemia, myeloproliferative disorders, myelodysplastic syndromes, pure red cell aplasia, paroxysmal nocturnal hemoglobinuria, Fanconi anemia, Thalassemia major, sickle cell anemia, severe combined immunodeficiency (SCID), Wiskott-Aldrich syndrome, hemophagocytic lymphohistiocytosis (HLH), mucopolysaccharidosis, Gaucher disease, metachromatic leukodystrophy, adrenoleukodystrophy, vascular disease, ischemia, and atherosclerosis. In some embodiments, the isolated hematopoietic stem cell is autologous to the subject in need thereof.

In yet another aspect, this disclosure also provides a method testing the toxicity of a compound on a population of hematopoietic stem cells. The method includes: (i) administering the compound to a population of the isolated hematopoietic stem cells described above; and (ii) comparing the response of the isolated hematopoietic stem cells exposed to the compound to the isolated hematopoietic stem cells not exposed to the compound.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows a reprogramming scheme for hematopoietic transcription factor (TF) screening. To assess the impact of other known hematopoiesis-inducing factors on GGF (GATA2, GFI1B, and FOS) reprogrammed cells, human adult dermal fibroblasts (HDFs, ScienCell) were first expanded and then split on day 1 of the reprogramming to a density of 150,000-300,000 per dish. Cells were then transduced three times and eventually split 1:2 into hematopoiesis-supporting media. Reprogrammed cells then had half media changes and were analyzed at day 30 for this experiment.

FIGS. 2A and 2B show that adding GFI1 to the hemogenic cocktail results in CD34⁺ progenitor expansion. FIG. 2A shows the experimental design of an N+1 and N−1 experiment using CD34 as a readout, to identify which TF acts in the hemogenic reprogramming. FIG. 2B shows GGF together with the individual factors from FGRS as well as FGRS with subtraction of one factor, both reveal that GFI1 acts as the causative factor that expands the CD34⁺ cells.

FIG. 3 shows cellular morphology of GGF (GATA2, GFI1B, and FOS) and 3GF ((GATA2, GFI1B, GFI1, and FOS) cells throughout reprogramming. Throughout 5 weeks of reprogramming, clear morphological differences can be seen in both GGF and 3GF cells. At later time points, however, a definite expansion of rounded hematopoietic-like cells in 3GF reprogrammed cells was observed.

FIGS. 4A and 4B show the induction of populations responsible for giving rise to early hematopoiesis. FIG. 4A shows staining of the day 30 human embryo for angiotensin-converting enzyme (ACE, also known as BB9) and CD49f. FIG. 4B shows quantification of relevant yields of these populations between GGF and 3GF reprogrammed cells.

FIGS. 5A, 5B, and 5C show expansion of various hematopoietic populations using 3GF. FIG. 5A shows representative flow plots and quantification of CD34⁺ throughout the reprogramming process between GGF and 3GF. FIG. 5B shows flow plots and quantification of both CD49f⁺CD34⁺ and BB9⁺CD34⁺ populations from GGF and 3GF reprogramming. FIG. 5C shows flow plots and quantification of CD49f⁺BB9⁺CD34⁺ progenitors between GGF and 3GF reprogramming.

FIGS. 6A, 6B, and 6C show induction of EPCR in GGF and 3GF cells. FIG. 6A shows live staining of GGF for EPCR throughout the reprogramming process. FIG. 6B shows EPCR live staining of 3GF cells. FIG. 6C shows representative flow plots of day 27 3GF cells for CD34, CD49f, and EPCR and quantification throughout reprogramming.

FIGS. 7A, 7B, and 7C show PCA plots for GGF and 3GF reprogrammed cells. FIG. 7A shows a comparison of dimension 1 and 2 between the GGF and 3GF RNAseq datasets, which reveals a strong separation purely based on the technical separation between the two experiments conducted to obtain these datasets (as separated by the dotted line). FIG. 7B shows a comparison of dimension 2 and 3 reveals many more interesting biological similarities and differences between each GGF and 3GF population. FIG. 7C shows hierarchical clustering of GGF and 3GF populations. After depleting dimension 1, which accounted for technical variance between the two different experiments, various clustering patterns between GGF and 3GF reprogrammed cells were observed. Box 1 highlights clustering of the D25 CD49f⁺CD34⁺ populations between GGF and 3GF. Box 2 highlights a close relationship between the uniquely derived 3GF D15 CD49f⁺CD34⁺ cells with GGF D25 CD49f⁺CD34⁻ cells.

FIGS. 8A, 8B, and 8C show DESeq2 MA plots with integrated published gene lists. FIG. 8A shows a comparative analysis of 3GF D15 CD49f⁺CD34⁻ to D25 CD49f⁺CD34⁺ populations using an HSC gene list from Notta et al., 2011 (Notta, F. et al. (2011). Science 333, 218-221). FIG. 8B show comparative analysis of 3GF D15 CD49f⁺CD34⁺ to D25 CD49f⁺CD34⁺ populations including a list of genes expressed in endothelial cells assembled from the existing literature. FIG. 8C comparative analysis of 3GF 15 CD49f⁺CD34⁻ to D15 CD49f⁺CD34⁺ populations using a both an HSC and endothelial gene list from Guibentif et al., 2017 (Guibentif, C. et al. (2017). Cell reports 19, 10-19).

FIG. 9 shows heat map and Gene Ontology (GO) term analysis of 3GF cells. Using 3GF D15CD49f⁺CD34⁻ expression as a baseline, genes up and downregulated in the disclosed more mature/differentiated cell populations relative to this baseline was found, leading to the identification of GO pathways pertaining to these gene expression changes.

FIG. 10 shows quantification of normalized read counts for select endothelial and hematopoietic genes. After DESeq2 normalization, specific genes in each of the four sequenced populations after 3GF reprogramming can be identified. Bar graphs represent genes commonly associated with endothelial identity and bar graphs represent genes commonly associated with hematopoietic identity are indicated.

FIG. 11 shows a long-term culture scheme to assess reprogrammed cell functional potential. TdT-HDFs transduced with 3GF and sorted on either day 15 or day 25 of reprogramming culture were seeded at initial densities of 20,000-30,000 cells per well into 12 well trays initially prepared with irradiated AFT024 monolayers. During the duration of the 5 weeks of LTC, cells were treated with DOX for 1, 2, 3, or 5 weeks and subsequently analyzed.

FIGS. 12A, 12B, and 12C show induction of functional cells after AFT024 co-culture. FIG. 12A shows seeding of CD49f⁺ 3GF cells onto AFT024 monolayers results in the derivation of cobblestone-like colonies after continued exposure to DOX for 5 weeks. FIG. 12B shows harvesting of colonies and seeding of colony-forming unit (CFU) assays results in the emergence of hematopoietic colonies composed of various hematopoietic cells in cytospins. FIG. 12C shows live staining of harvested CFU colonies reveals CD45⁺ cells composed primarily of CD235a⁺ erythroid cells, with a large population of CD14⁺ myeloid cells as well.

FIGS. 13A, 13B, 13C, 13D, and 13E show that LDA on AFT024 monolayers allows determination of stem cell frequency. FIG. 13A shows a scheme for plating reprogrammed GGF or 3GF cells in 96 well trays to assess stem cell frequency. For reprogrammed cells, row A receives 10,000 and serially diluted by 50% to row H that has 78.125 cells per well. For Lin⁻CD34⁺ CB HSCs, row A begins with 1000 cells, and after serial dilutions row H has 7.8125 cells per well. FIG. 13B shows representative images to demonstrate + and − colonies. FIG. 13C shows LDA plot for GGF and 3GF reprogrammed cells. FIG. 13D shows LTC and cytospin images of Lin_CD34⁺ CB HSCs after 5 weeks of LTC on AFT. FIG. 13E shows LDA plot for Lin⁻CD34⁺ CB HSCs.

FIG. 14 shows isolation of relevant hematopoietic populations from 3GF reprogrammed cells after 5 weeks on Gelatin (G) or AFT024 (A). FACS quantification of samples isolated after 5 weeks of AFT024 LTC.

FIGS. 15A, 15B, 15C, 15D, and 15E show functional analyses of CB HSCs after LTC⁻ IC. FIG. 15A shows the number and type of CFU from 200 Lin-CD34+ after 5 weeks of LTC on AFT024.FIG. 15B shows quantification of emerging colonies. FIG. 15C shows representative flow plots of harvested CFU colonies. FIG. 15D shows quantification of relevant populations from panel C. FIG. 15E shows secondary LDA data for Lin⁻CD34⁺ CB HSCs after 5 weeks of AFT024 LTC.

FIGS. 16A, 16B, and 16C show short-term multilineage engraftment of day 15CD49f+ 3GF cells. FIG. 16A shows a scheme for 3GF reprogramming and transplants. IH=Intrahepatic. FIG. 16B shows flow plots for mouse vs. human CD45⁺ cells. FIG. 16C shows multilineage reconstitution from the hCD45⁺ population from Mouse ID #1 on week 8.

### DETAILED DESCRIPTION OF THE INVENTION

The disclosed methods for programming human somatic cells into hematopoietic stem cells (HSCs) are based on an unexpected discovery that inducing expression of a combination of GATA2, GFI1B, GFI1, and FOS transcription factors (TFs), termed “3GF TF cocktail,” as well as co-culture with AFT024 stroma cells, results in a high yield of cells that possess key characteristics of HSCs, such as multilineage functionality in vivo and in vitro, multipotency, and self-renewal.

A replenishable source of engraftable, autologous human blood cells can provide a potential foundation to study and ultimately cure a multitude of hematologic disorders. Previous reprogramming strategies, in both human induced pluripotent stem cells (iPSCs) and somatic cells, remain limited in the identity of their final derived cells or have practical issues with either their starting cell populations or transcription factor (TF) cocktails. Thus far, the low efficiency and poor engraftment capabilities of cells derived through iPSC differentiation restricts the utility of this method. This disclosure provides an optimized hemogenic induction process without going through pluripotency to yield cells that parallel endogenous HSCs in their cell surface phenotype, gene expression profile, and functional potential. The new 3GF TF cocktail, as well as co-culture on AFT024 stroma, improves the yield and functional output of the generated cells.

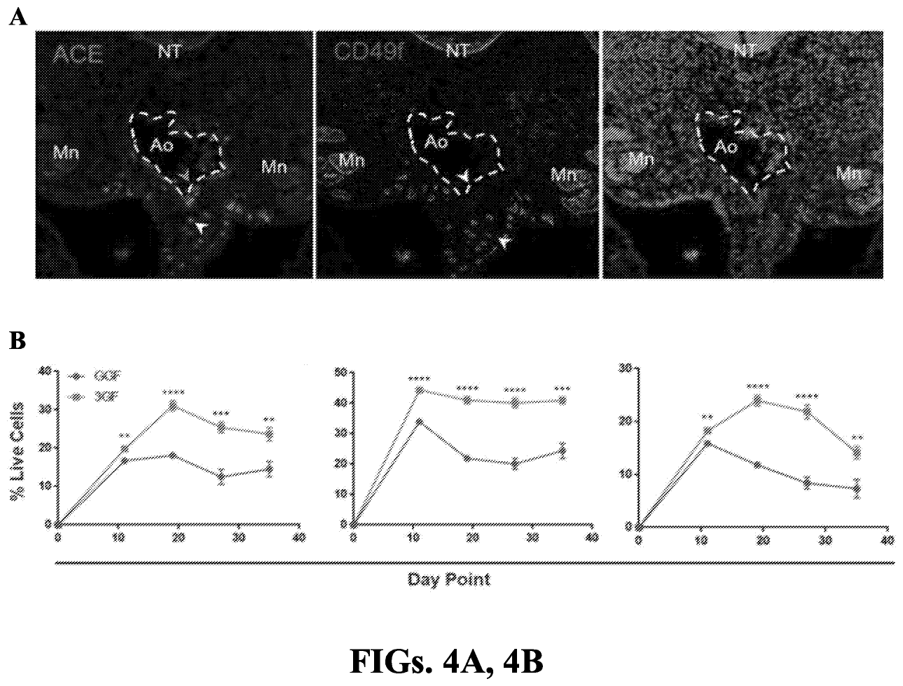

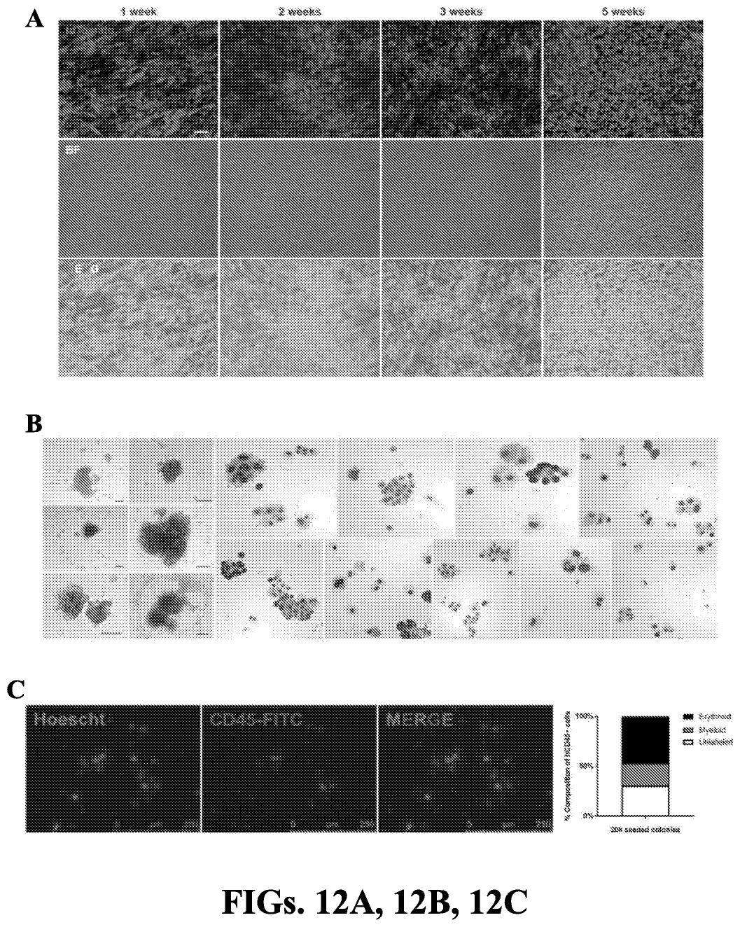

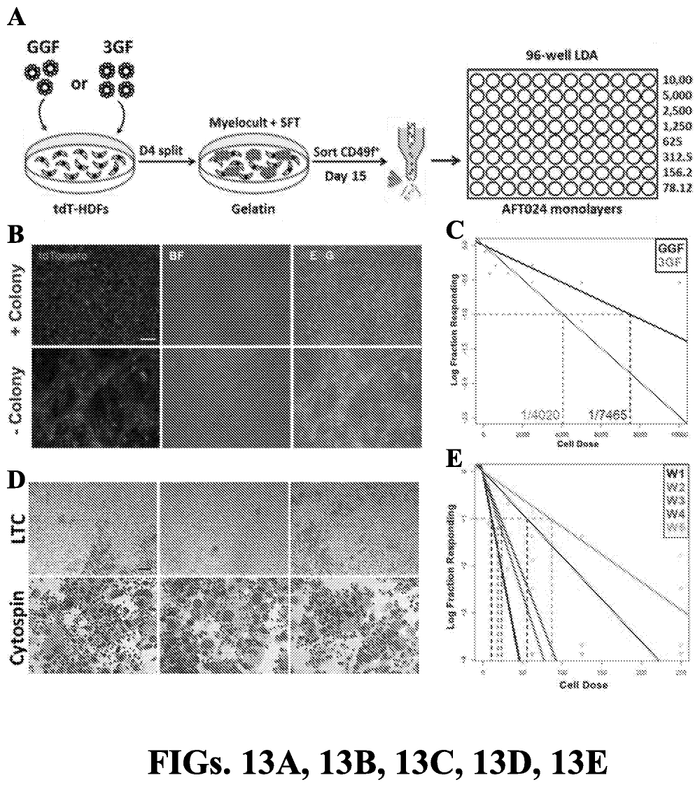

This disclosure demonstrates that the same developmental program in both mouse and human fibroblasts can be induced to derive hematopoietic cells. Addition of GFI1 to the GGF reprogramming cocktail (including GATA2, GFI1B, and FOS transcription factors) yields significantly expanded progenitors assayed by any cell surface profile selected (FIGS. 4-6). Mechanistically, this inclusion highlights the importance of the axis formed by GFI1 and GFI1B in regulating human hematopoiesis and EHT via RUNX and other pathways. This disclosure also demonstrates in vitro maturation of both GGF and 3GF cells on AFT024 stroma results in the derivation of cells with clonogenic potential, with 3GF reprogramming yielding more functional cells both qualitatively and quantitatively (FIGS. 12-13).

This disclosure also provides an in vitro platform for drug testing and hematopoietic disease modeling for identification of treatments for hematopoietic disorders and avenues for autologous HSC transplants.

### I. Programming Human Somatic Cells into Hematopoietic Stem Cells

The disclosure provides a method for programming a host cell into a hematopoietic stem cell. The method includes introducing into the host cell a combination of transcription factors, including GATA binding protein 2 (GATA2), growth factor independent 1B (GFI1B), growth factor independent 1 (GFI1), and FBJ osteosarcoma oncogene (FOS).

Also within the scope of this disclosure are the variants and homologs with significant identity to the transcription factors (TFs) discribed herein (i.e., GATA2, GFI1B, GFI1, FOS). For example, such variants and homologs may have sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity over the sequences of the TFs discribed herein.

“GATA binding protein 2 (GATA2)” or a homolog thereof, is the TF introduced into a host cell in the methods described herein. GATA2 is a member of the GATA family of zinc-finger transcription factors, named for the consensus nucleotide sequence they bind in the promoter regions of target genes. The encoded protein plays an essential role in regulating transcription of genes involved in the development and proliferation of hematopoietic and endocrine cell lineages. The disclosure includes, but is not limited to, GATA2 provided in GenBank accession numbers NM_008090.5 (mouse) and M68891.1 (human).

“Growth factor independent 1B (GFI1B),” or a homolog thereof, is the TF introduced into a host cell in the methods described herein. GFI1B is a transcriptional repressor and a target of E2A. GFI1B promotes growth arrest and apoptosis in lymphomas. GFI1B expression in primary T-lymphocyte progenitors is dependent on E2A, and excess GFI1B prevents the outgrowth of T lymphocyte progenitors in vitro. GFI1B represses expression of GATA3, a transcription factor whose appropriate regulation is required for survival of lymphomas and T-lymphocyte progenitors. The disclosure includes, but is not limited to, GFI1B provided in GenBank accession numbers AF017275.1 (mouse) and NM_004188.4 (human).

“Growth factor independent 1 (GFI1),” or a homolog thereof, a nuclear zinc finger protein that functions as a transcriptional repressor. This protein plays a role in diverse developmental contexts, including hematopoiesis and oncogenesis. It functions as part of a complex along with other cofactors to control histone modifications that lead to silencing of the target gene promoters. Mutations in this gene cause autosomal dominant severe congenital neutropenia, and also dominant nonimmune chronic idiopathic neutropenia of adults, which are heterogeneous hematopoietic disorders that cause predispositions to leukemias and infections. The disclosure includes, but is not limited to, GFI1 provided in GenBank accession numbers NM_010278.2 (mouse) and NM_005263.4 (human).

The “FBJ osteosarcoma oncogene or c-Fos or FOS” is the TF introduced into a host cell in the methods described herein. c-Fos is a protein encoded by the FOS gene. FOS is a cellular proto-oncogene belonging to the immediate early gene family of transcription factors. c-Fos has a leucine-zipper DNA binding domain and a transactivation domain at the C-terminus. Transcription of c-Fos is upregulated in response to many extracellular signals, e.g., growth factors. The disclosure includes, but is not limited to, FOS provided in GenBank accession numbers NM010234.2 (mouse) and NM005252.3 (human).

The expression of the TFs can be induced by introducing one or more expression vectors carrying nucleic acids encoding the TFs. To construct the expression vector for the TFs, a nucleic acid molecule encoding a TF polypeptide or fragment thereof is inserted into the proper site of the vector (e.g., operably linked to a promoter). The expression vector is introduced into a selected host cell for amplification and/or polypeptide expression, by well-known methods such as transfection, transduction, infection, electroporation, microinjection, lipofection or the DEAE-dextran method or other known techniques. These methods and other suitable methods are well known to the skilled artisan.

Many vectors useful for transferring exogenous genes into target mammalian cells are available. The vectors may be episomal, e.g., plasmids, virus-derived vectors such cytomegalovirus, adenovirus, etc., or may be integrated into the target cell genome, through homologous recombination or random integration, e.g., retrovirus-derived vectors such MMLV, HIV-1, ALV, etc. Lentiviral vectors such as those based on HIV or FIV gag sequences can be used to transfect non-dividing cells, such as the resting phase of human stem cells (see Uchida et al. (1998) P.N.A.S. 95(20): 1 1939-44).

Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g., 24-73 hours, or for at least two weeks, and may be allowed to grow for five weeks or more, before analysis. Commonly used retroviral vectors are “defective,” i.e., unable to produce viral proteins required for productive infection. Replication of the vector requires growth in the packaging cell line. The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g., MMLV, are capable of infecting most murine and rat cell types. Ecotropic packaging cell lines include BOSC23 (Pear et al. (1993) P.N.A. S. 90:8392-8396). Retroviruses bearing amphotropic envelope protein, e.g., 4070A (Danos et al. (1988) PNAS 85:6460-6464), are capable of infecting most mammalian cell types, including human, dog, and mouse. Amphotropic packaging cell lines include PA12 (Miller et al. (1985) Mol. Cell. Biol. 5:431-437); PA317 (Miller et al. (1986) MpJ. CelL BioL 6:2895-2902) GRIP (Danos et al. (1988) PNAS 85:6460-6464). Retroviruses packaged with xenotropic envelope protein, e.g., AKR env, are capable of infecting most mammalian cell types, except murine cells. The vectors may include genes that must later be removed, e.g., using a recombinase system such as Cre/Lox, or the cells that express them destroyed, e.g., by including genes that allow selective toxicity such as herpesvirus TK, bcl-xs, etc. Suitable inducible promoters are activated in a desired target cell type, either the transfected cell or progeny thereof.

In some embodiments, the nucleic acid(s) encoding the described TFs may be introduced by using one or more viral vectors. In one example, the nucleic acids encoding the described TFs can be carried on a single viral vector. In another example, one or more nucleic acids encoding the described TFs can be carried on separate viral vectors. “Viral vector” as disclosed herein refers to, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as an endothelial cell or hematopoietic cell in culture.

The nucleic acids encoding the described TFs may be carried on separate viral vectors, respectively, or on a single viral vector. In some embodiments, a retroviral (e.g., pBABE-puro) or lentiviral vector (e.g., pFUW-TetO) is used as the expression vector for introducing the various TFs described herein.

Host cells may include, without limitation, various cell types in the body. For example, host cells may include somatic cells, such as fibroblasts (e.g., human dermal fibroblasts), epithelial cells, bone marrow cells, differentiated hematopoietic cells (e.g., B and T lymphocytes), macrophages, hematopoietic progenitor cells, and peripheral blood mononuclear cells (PBMCs).

Host cells may include cells that are derived primarily from endoderm, such as exocrine secretory epithelial cells, hormone-secreting cells, epithelial cells lining internal body cavities, and ciliated cells. Examples of such cells include, but are not limited to, salivary gland mucous cells, salivary gland serous cells, Von Ebner's gland cells, mammary gland cells, lacrimal gland cells, ceruminous gland cells, eccrine sweat gland dark cells, eccrine sweat gland clear cells, apocrine sweat gland cells, gland of Moll cell, sebaceous gland cells, Bowman's gland cells, Brunner's gland cells, seminal vesicle cells, prostate gland cells, bulbourethral gland cells, Bartholin's gland cells, gland of Littre cells, endometrium cells, goblet cells, mucous cells, zymogenic cells, oxyntic cells, acinar cells, Paneth cells, Type II pneumocytes, Clara cells, pituitary cells (e.g., somatotropes, lactotropes, thyrotropes, gonadotropes, and corticotropes), magnocellular neurosecretory cells, intestinal cells, respiratory tract cells, thyroid gland cells, thyroid epithelial cells, parafollicular cells, parathyroid gland cells, chief cells, oxyphil cells, adrenal gland cells, chromafin cells, Leydig cells, theca cells, granulosa cells, corpus luteum cells, juxtaglomerular cells, macular cells, macula densa cells, peripolar cells, mesangial cells, endothelial fenestrated cells, endothelial continuous cells, endothelial splenic cells, synovial cells, serosal cells, squamous cells, columnar cells, dark cells, vestibular membrane cells, basal cells, marginal cells, cells of Claudius, cells of Boettcher, choroid plexus cells, ciliary epithelial cells, corneal endothelial cells, Peg cells, respiratory tract ciliated cells, oviduct ciliated cells, uterine endometrial ciliated cells, rete testis ciliated cells, ductulus deferens ciliated cells, and ciliated ependymal cells.

Host cells may include cells that are derived primarily from ectoderm, such as keratinizing epithelial cells, wet stratified barrier epithelial cells, sensory transducer cells of the nervous system, autonomic neurons, sense organ and peripheral neuron supporting cells, central nervous system neurons and glial cells, and lens cells. Such cells include, but are not limited to, epidermal keratinocytes, epidermal basal cells, keratinocytes, nail bed basal cells, hair shaft cells, hair root sheath cells, hair matrix cells, surface epithelial cells of stratified squamous epithelium, basal epithelial cells, urinary epithelial cells, auditory inner and outer hair cells of organ of Corti, basal cells of olfactory epithelium, cold-sensitive primary sensory neurons, heat-sensitive primary sensory neurons, Merkel cells, olfactory receptor neurons, pain-sensitive primary sensory neurons, photoreceptor cells of the retina (e.g., rod cells, blue-sensitive cone cells, green-sensitive cone cells, and red-sensitive cone cells), proprioceptive primary sensory neurons, touch-sensitive primary sensory neurons, type I carotid body cells, type II carotid body cells, type I and type II hair cells of vestibular apparatus of ear, type I taste bud cells, cholinergic neurons, adrenergic neurons, peptidergic neurons, inner and outer pillar cells of organ of Corti, inner and outer phalangeal cells of organ of Corti, border cells of organ of Corti, Hense cells of organ of Corti, vestibular apparatus supporting cells, taste bud supporting cells, olfactory epithelium supporting cells, Schwann cells, satellite cells, enteric glial cells, astrocytes, neurons, oligodendrocytes, Spindle neurons, anterior lens epithelial cells, and crystallin-containing lens fiber cells.

Host cells may include cells that are derived primarily from mesoderm, such as metabolism and storage cells, barrier function cells, kidney cells, extracellular matrix cells, contractile cells, blood, and immune system cells, pigment cells, germ cells, nurse cells, and interstitial cells. Such cells include, but are not limited to, hepatocytes, adipocytes (e.g., white fat cells and brown fat cells), liver lipocytes, glomerulus parietal cells, glomerulus podocytes, proximal tubule brush border cells, Loop of Henle thin segment cells, distal tubule cells, collecting duct cells, type 1pneumocytes, centroacinar cells, nonstriated duct cells (e.g., principal cells and intercalated cells), duct cells, intestinal brush border cells, exocrine gland striated duct cells, gall bladder epithelial cells, ductus deferens nonciliated cells, epididymal prinicipal and basal cells, ameloblast epithelial cells, planum semilunatum epithelial cells, Organ of Corti interdental epithelial cells, loose connective tissue fibroblasts, corneal fibroblasts, tendon fibroblasts, bone marrow reticular tissue fibroblasts, nonepithelial fibroblasts, pericytes, nucleus pulposus cells, cementoblasts, cementocytes, odontoblasts odontocytes, hyaline cartilage chondrocytes, fibrocartilage chondrocytes, elastic cartilage chondrocytes, osteoblasts, osteocytes, osteoprogenitor cells, hyalocytes, stellate cells (i.e., of the ear, liver, and pancreas), skeletal muscle cells (e.g., red skeletal muscle cells (slow), white skeletal muscle cells (fast), intermediate skeletal muscle cell, nuclear bag cells of muscle spindle, and nuclear chain cell of muscle spindle), satellite cells, heart muscle cells (e.g., ordinary heart muscle cells, nodal heart muscle cells, and Purkinje fiber cells), smooth muscle cell, myoepithelial cells, erythrocytes, megakaryocytes, monocytes, connective tissue macrophages, epidermal Langerhans cells, osteoclasts, dendritic cells, microglial cells, neutrophil granulocytes, eosinophil granulocytes, basophil granulocytes, mast cells, helper T cells, suppressor T cells, cytotoxic T cells, natural killer T cells, B cells, natural killer cells, reticulocytes, stem cells and committed progenitors of the blood and immune system, melanocytes, retinal pigmented epithelial cells, oogonium, oocytes, spermatids, spermatocytes, spermatogonium cell, spermatozoan, ovarian follicle cells, Sertoli cells, thymus epithelial cells, and interstitial kidney cells.

HSCs are the stem cells that give rise to other blood cells, including the myeloid and lymphoid lineages of blood cells. This process is called hematopoiesis that occurs in the red bone marrow. HSCs are non-adherent, and rounded, with a rounded nucleus and low cytoplasm-to-nucleus ratio. They are characterized by their extensive self-renewal capacity and pluripotency. HSCs can replenish all blood cell types (i.e., are multipotent) and self-renew. A small number of HSCs can expand to generate a very large number of daughter HSCs. This phenomenon is used in bone marrow transplantation, when a small number of HSCs reconstitute the hematopoietic system.

HSCs lack expression of mature blood cell markers and are thus, called Lin-. Lack of expression of lineage markers is used in combination with detection of several positive cell-surface markers to isolate HSCs. For example, HSCs are determined as CD34⁺, CD59⁺, CD90/Thy1⁺, CD38^{low/-}, c-Kit^{-/low}, and Lin⁻. Mouse Hematopoietic Stem Cells are considered CD34^{low/-}, SCA-1⁺, CD90/Thy1^{+/low}, CD38⁺, c-Kit⁺, and Lin⁻. Detecting the expression of these marker panels allows separation of specific cell populations via techniques like fluorescence-activated cell sorting (FACS). In addition, HSCs are characterized by their small size and low staining with vital dyes such as rhodamine 123 (rhodamine^{lo}) or Hoechst 33342 (side population).

In another aspect, this disclosure also provides a method of maintaining/expanding derived HSCs by co-culturing HSCs with other cells, including, without limitation, stromal cells. In some embodiments, stromal cells may include AFT024, derived from murine fetal liver (FL). This disclosure demonstrates that stromal cell line AFT024 can support both mouse and human hematopoiesis in vitro. AFT024 shown to express key signals for sustaining hematopoiesis, such as DLK1—which constitutes a non-canonical ligand for NOTCH—and DPT. AFT024 supports the ex vivo maintenance of human CD34⁺CD38⁻ HPCs significantly more efficiently than other human-derived cell lines in a contact-dependent manner, highlighting the plethora of signals these cells specifically express to support hematopoiesis in vitro. This disclosure also demonstrates that in vitro maturation of both GGF and 3GF cells on AFT024 stroma results in the derivation of cells with clonogenic potential, with 3GF reprogramming yielding more functional cells both qualitatively and quantitatively.

### H. Isolated Hematopoietic Stem Cells, Compositions, And Kits

A major challenge for researchers using HSCs is their identification and isolation from larger pools of cells. It is estimated that HSCs represent approximately 1 in 10,000 cells of the bone marrow and 1 in 100,000 cells in the blood. Thus, this disclosure also provides methods for identifying and isolating HSCs. HSCs can be identified by their small size, large nuclear to cytoplasmic ratio, and other properties. Alternatively, HSCs can be identified by screening the cell for expression of a hemogenic endothelial cell marker or a multipotent HSC marker, or by uptake of acetylated low-density lipoprotein (acLDL). In some examples, methods for identifying an HSC are performed by labeling the cell with a marker that appears on the surface of the cell. Cell surface markers are widely used according to methods known in the art to identify cells, and HSCs express a wide variety and combination of markers. For example, markers for human HSCs include, without limitation, CD31, CD34, CD38^{lo/-, CD}41, CD43, CD45, CD49f, Thy1/CD90, CD105, CD117/c-kit, CD133, CD143, CD150, CD201, Sca-1, Tie2, VE-Cadherin, KDR/FLK1, Flk-2/Flt3, and CXCR4. In some embodiments, such cell markers may be tagged with monoclonal antibodies bearing a fluorescent label and analyzed or isolated with fluorescence-activated cell sorting (FACS). In some embodiments, acLDL and lectin may be coupled to fluorescent markers and bind on the cell surface.

Thus, in one aspect, the method may include screening the cell for expression of a hemogenic endothelial cell marker or a hematopoietic stem cell marker, such as CD31, CD34, CD38^{lo/-}, CD41, CD43, CD45, CD49f, Thy1/CD90, CD105, CD117/c-kit, CD133, CD143, CD150, CD201, Sca-1, Tie2, VE-Cadherin, KDR/FLK1, Flk-2/Flt3, and CXCR4.

Many of these cell markers are commercially available, such as D133-APC, SCA-1-PE, Tie2-PE, CD11b-APC, CD31-PE, CD41-APC; and VE-cadherin (eBioscience, San Diego, Calif.); CD45-PE, Flk1-PE, and CD43-APC (BD Biosciences, Sparks, Md.); c-kit-APC (BioLegend®, San Diego, Calif.); and acLDL-Dil (Biomedical Technologies, Inc., Stoughton, Mass.). Marker expression profiles on the GFP⁺ cells can be analyzed by analytical flow cytometry and FACS.

HSCs are negative for the markers (e.g., Lin⁻) that are used for detection of lineage commitment. Thus, in some aspects, the methods of the disclosure include screening a cell for lack of expression of a differentiated hematopoietic lineage (lin) marker, i.e., screening for a Lin⁻ cell. A lin⁻ marker may include, without limitation, CD4, CD5, CD8, CD45RA/B220, Gr-1/Ly-6G/C, and Ter119.

After screening HSCs for the expression of appropriate hematopoietic markers, HSCs can be isolated and/or purified. Cells can be isolated by any method known in the art, e.g., FACS. In some examples, the HSCs are isolated and frozen with a cryo-protectant. Methods of freezing cells are well known in the art, and all such methods of freezing cells are included for use in this disclosure. Isolated HSCs are available for treatment of a subject in need thereof, for freezing, for further experimentation, or for further cell culture.

HSCs may be cultured using standard media well known in the art. The media usually contains all nutrients necessary for the growth and survival of the cells. In some examples, additional nutrients are supplemented as needed. Suitable media for culturing eukaryotic cells include, without limitation, Roswell Park Memorial Institute medium 1640 (RPMI 1640), Minimal Essential Medium (MEM), Dulbecco's Modified Eagle Medium (DMEM), and Myelocult Medium (Stem Cell Technologies, M5300 and H5100), all of which, in some instances, are supplemented with serum and/or growth factors as indicated by the particular cell line or type being cultured.

In some examples, an antibiotic or other compounds useful for selective growth of transduced or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, and neomycin.

In some examples, the transduced cells are cultured on gelatin or co-cultured on irradiated cells of another cell line with or without a combination of cytokines. In some aspects, the HSCs are cultured in optimized conditions in a serum-free culture medium.

In another aspect, this disclosure provides isolated hematopoietic stem cells obtained by the methods described above and a composition comprising isolated hematopoietic stem cells an appropriate vehicle for delivery of the cells to a subject in need thereof. In addition, the disclosure includes a composition comprising such isolated HSCs and a cryo-protectant.

Pharmaceutical compositions are also included in the disclosure. In some aspects, a pharmaceutical composition of the disclosure comprises a population of HSCs and a pharmaceutically acceptable diluent, carrier or medium. The phrase “pharmaceutically acceptable” refers to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like. In all aspects, the carriers of the disclosure have to be appropriate for delivery with live cells.

HSCs are generally administered intravenously by routine clinical practice. The dose is dependent upon the source of the stem cells (e.g., bone marrow, mobilized peripheral blood cells, and cord blood) and the donor (e.g., autologous and allogeneic, including HLA-matched/mismatched). A typical dose includes, but is not limited to, a dose in the range of 5 to 10⁶ cells/kg.

Also within the scope of this disclosure is a kit comprising the isolated hematopoietic stem cell or the composition described above. The kit may further include instructions for administrating the isolated hematopoietic stem cell or the composition and optionally an adjuvant. In another aspect, this disclosure also provides a kit for hemogenic reprogramming, including one or more recombinant expression viruses or virus-like particles that comprise a nucleic acid encoding GATA2, a nucleic acid encoding GFI1B, a nucleic acid encoding GFI1, and a nucleic acid encoding FOS. The kit may further include instructions for introducing the recombinant expression viruses or virus-like particles described above into a subject in need thereof and optionally an adjuvant.

HSCs are stem cells that form blood and immune cells. HSCs are ultimately responsible for the constant renewal of blood and produce up to billions of new blood cells each day. HSCs are multipotent stem cells that give rise to all the blood cell types from the myeloid (including, but not limited to, monocytes and macrophages, neutrophils, basophils, eosinophils, erythrocytes, megakaryocytes/platelets, and dendritic cells), and lymphoid lineages (including, but not limited to, T-cells, B-cells, and NK-cells). Thus, in another aspect, this disclosure provides blood, cellular and acellular blood components, blood products or hematopoietic stem cells comprising the isolated hematopoietic cells described above.

### III. Methods of use and Treatment

The production of HSCs allows for the study of the cellular and molecular biology of events of human and mouse development, generation of differentiated cells for use in transplantation (e.g., autologous or allogeneic transplantation), treating diseases (e.g., any described herein), in vitro drug screening or drug discovery, disease modeling, and cryopreservation.

### A. Transplantation and Treatment of Disease

HSCs of the disclosure are used in hematopoietic stem cell transplantation (HSCT). HSCT is a procedure in which multipotent progenitor cells, such as HSCs, blood stem cells, or umbilical cord blood capable of reconstituting normal bone marrow function, are administered to a patient. This procedure is often performed as part of therapy to eliminate a bone marrow infiltrative process, such as leukemia, or to correct congenital immunodeficiency disorders. Recent work in this field has expanded its use to allow patients with cancer to receive higher doses of chemotherapy than the bone marrow can usually tolerate; bone marrow function is then salvaged by replacing the marrow with previously harvested stem cells.

In another aspect, this disclosure also provides a method of engraftment or cell replacement for autologous or non-autologous transplantation in a subject in need thereof comprising transferring to the subject the isolated hematopoietic cells described above.

In another aspect, this disclosure also provides a method for treating a subject who suffers from a condition or a disease that would benefit from HSCT, thus making them a candidate for HSCT. The method includes administering to the subject a therapeutically effective amount of the isolated hematopoietic stem cells described above. In some embodiments, the isolated hematopoietic stem cell is autologous to the subject in need thereof. The condition or disease may include cancer, a congenital disorder, and vascular disease.

In some embodiments, the subject may suffer from multiple myeloma or leukemia and undergo prolonged treatment with, or are already resistant to, chemotherapy. In some aspects, candidates for HSCT include pediatric cases where the patient has an inborn defect such as severe combined immunodeficiency or congenital neutropenia with defective stem cells, and also children or adults with aplastic anemia who have lost their stem cells after birth. Other conditions that benefit from HSCT include, but are not limited to, sickle-cell disease, myelodysplastic syndrome, neuroblastoma, lymphoma, Ewing's Sarcoma, Desmoplastic small round cell tumor, chronic granulomatous disease, and Hodgkin's disease. More recently non-myeloablative, or so-called “mini-transplant,” procedures have been developed that require smaller doses of preparative chemotherapy and radiation. This has allowed HSCT to be conducted in the elderly and other patients who would otherwise be considered too weak to withstand a conventional treatment regimen.

HSCs of the disclosure have the potential to differentiate into a variety of cell types including, but not limited to, all cell types of a hematopoietic lineage. Accordingly, HSCs of the disclosure can be transplanted into a subject to treat a number of conditions or diseases which could benefit from HSCT including, but not limited to, cancer, congenital disorders, or vascular disease. More specific conditions or diseases which could benefit from HSCT include, but are not limited to, multiple myeloma, leukemia, congenital neutropenia with defective stem cells, aplastic anemia, myelodysplastic syndrome, neuroblastoma, lymphoma, Ewing's Sarcoma, Desmoplastic small round cell tumor, chronic granulomatous disease, non-Hodgkin's lymphoma, Hodgkin's disease, acute myeloid leukemia, neuroblastoma, germ cell tumors, systemic lupus erythematosus (SLE), systemic sclerosis, amyloidosis, acute lymphoblastic leukemia, chronic myeloid leukemia, chronic lymphocytic leukemia, myeloproliferative disorders, myelodysplastic syndromes, pure red cell aplasia, paroxysmal nocturnal hemoglobinuria, Fanconi anemia, Thalassemia major, sickle cell anemia, severe combined immunodeficiency (SCID), Wiskott-Aldrich syndrome, hemophagocytic lymphohistiocytosis (HLH), mucopolysaccharidosis, Gaucher disease, metachromatic leukodystrophy, adrenoleukodystrophy, ischemia, and atherosclerosis.

The HSCs induced by the disclosed methods may be administered in any physiologically acceptable excipient (e.g., William's E medium), where the cells may find an appropriate site for survival and function (e.g., organ reconstitution). The cells may be introduced by any convenient method (e.g., injection, catheter, or the like). The cells may be introduced to the subject (i.e., administered into the individual) via any of the following routes: parenteral, subcutaneous, intravenous, intracranial, intraspinal, intraocular, or into the spinal fluid. The cells may be introduced by injection (e.g., direct local injection), catheter, or the like. Examples of methods for local delivery (e.g., delivery to the liver) include, e.g., by bolus injection, e.g., by a syringe, e.g., into a joint or organ; e.g., by continuous infusion, e.g., by cannulation, e.g., with convection (see, e.g. US Application No. 20070254842); or by implanting a device upon which the cells have been reversibly affixed (see, e.g., US Application Nos. 20080081064 and 20090196903). In some examples, HSCs are administered into an individual by ultrasound-guided liver injection. In this way, cells can be placed directly into a bloodstream (e.g., in humans, or even in mice using a small animal ultrasound system). Brightness mode (B-mode) can be used to acquire two-dimensional images for an area of interest with a transducer and cells can be injected in solution (e.g., 100 ml to 300 ml, e.g., 200 ml of, for example, William's E medium) into one site or many sites (e.g., 1-30 sites) in the blood using, for example, a 30 gauge needle.

The number of administrations of treatment to a subject may vary. Introducing cells into an individual may be a one-time event; but in certain situations, such treatment may elicit improvement for a limited period of time and require an on-going series of repeated treatments. In other situations, multiple administrations of hematopoietic stem cells may be required before an effect is observed. As will be readily understood by one of ordinary skill in the art, the exact protocols depend upon the disease or condition, the stage of the disease and parameters of the individual being treated.

A “therapeutically effective amount” or “dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of hematopoietic stem cells is an amount that is sufficient, when administered to (e.g., transplanted into) the individual, to palliate, ameliorate, stabilize, reverse, prevent, slow or delay the progression of the disease state (e.g., blood cell disorder) by, for example, providing functions normally provided by a subject with healthy blood.

In some embodiments, a therapeutically effective dose of HSCs is in a range of from about 1×10 cells to about 1×10¹⁰ cells (e.g, from about 5×10 cells to about 1×10¹⁰ cells, from about 1×10² cells to about 1×10¹⁰ cells, from about 5×10² cells to about 1×10¹⁰ cells, from about 1×10³ cells to about 1×10¹⁰ cells, from about 5×10³ cells to about 1×10¹⁰ cells, from about 1×10⁴ cells to about 1×10¹⁰ cells, from about 5×10⁴ cells to about 1×10¹⁰ cells, from about 1×10⁵ cells to about 1×10¹⁰ cells, from about 5×10⁵ cells to about 1×10¹⁰ cells, from about 1×10⁶ cells to about 1×10¹⁰ cells, from about 5×10⁶ cells to about 1×10¹⁰ cells, from about 1×10⁷ cells to about 1×10¹⁰ cells, from about 5×10⁷ cells to about 1×10¹⁰ cells, from about 1×10⁸ cells to about 1×10¹⁰ cells, from about 5×10⁸ cells to about 1×10¹⁰ cells, from about 1×10⁹ cells to about 1×10¹⁰ cells, from about 5×10⁹ cells to about 1×10¹⁰ cells.

HSCs of this disclosure can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

HSCs of the disclosed methods may be genetically altered in order to introduce genes useful in the differentiated hepatocytes, e.g., repair of a genetic defect in an individual, selectable marker, etc. Cells may also be genetically modified to enhance survival, control proliferation, and the like. Cells may be genetically altered by transfection or transduction with a suitable vector, homologous recombination, or other appropriate technique, so that they express a gene of interest. In some embodiments, a selectable marker is introduced, to provide for greater purity of the desired differentiating cell. The cells of this disclosure can also be genetically altered in order to enhance their ability to be involved in tissue regeneration or to deliver a therapeutic gene to a site of administration.

### B. Disease Modeling

HSCs can be generated to model and study hematological diseases in vitro. HSCs of the disclosure, in various aspects, are generated from subjects with conditions or diseases including, but not limited to, multiple myeloma, non-Hodgkin's lymphoma, Hodgkin's disease, acute myeloid leukemia, neuroblastoma, germ cell tumors, systemic lupus erythematosus (SLE), systemic sclerosis, amyloidosis, acute lymphoblastic leukemia, chronic myeloid leukemia, chronic lymphocytic leukemia, myeloproliferative disorders, myelodysplastic syndromes, aplastic anemia, pure red cell aplasia, paroxysmal nocturnal hemoglobinuria, Fanconi anemia, Thalassemia major, sickle cell anemia, severe combined immunodeficiency (SCID), Wiskott-Aldrich syndrome, hemophagocytic lymphohistiocytosis (HLH), inborn errors of metabolism, e.g., mucopolysaccharidosis, Gaucher disease, metachromatic leukodystrophies, adrenoleukodystrophies, and a variety of vascular disorders including, but not limited to, ischemia and atherosclerosis. The disclosure, therefore, provides a new technology so that disease-specific HSCs are generated for disease modeling and research. HSCs can be differentiated to any cell type of hematopoietic lineages to dissect in vitro the molecular mechanisms of hematological malignancies.

### C. Toxicology Screening

In various aspects, HSCs of the disclosure are used in toxicity screening. A method testing the toxicity of a compound on a population of hematopoietic stem cells may include: (i) administering the compound to a population of the isolated hematopoietic stem cells described above; and (ii) comparing the response of the isolated hematopoietic stem cells exposed to the compound to the isolated hematopoietic stem cells not exposed to the compound.

For example, assays are used to test the potential toxicity of compounds on the HSCs or the differentiated progeny thereof. In one example, where the HSCs are differentiated into a hematopoietic lineage, hematopoietic stem cells and progenitor assays can be used to investigate growth and differentiation of cells in response to positive and negative regulators of hematopoiesis. These assays provide the opportunity to assess the potential toxicity of compounds on specific hematopoietic (e.g., myeloid, erythroid) cell populations. For example, some assays to assess the toxicity of compounds on hematopoietic cells have been described by Van Den Heuvel et al. (Cell Biol. Toxicol. 17: 107-16, 2001), Kumagai et al. (Leukemia 8:1116-23, 1994), and in U.S. Patent Application Publication Nos. US2004/0029188, US2008/0248503, and US2011/0008823.

Other approaches include, prior to applying the drug, transforming the cells with a promoter activated by metabolic or toxicologic challenge operably linked to a reporter gene. Exemplary promoters include those which respond to apoptosis, respond to DNA damage, respond to hyperplasia, respond to oxidative stress, are upregulated in liver toxicity, are responsive to receptors that act in the nucleus, upregulate hepatocyte enzymes for drug metabolism, are from genes which are deficient in particular disease conditions, and genes which regulate synthesis, release, metabolism, or reuptake of neurotransmitters. See, for example, the methods and exemplary promoters in U.S. Patent Application Publication No. 2006/0292695.

In some examples, HSC progeny of a selected cell type can be cultured in vitro and used for the screening of potential therapeutic compositions. These compositions can be applied to cells in culture at varying dosages, and the response of the cells monitored for various time periods. Physical characteristics of the cells can be analyzed, for example, by observing cell growth with microscopy. The induction of expression of new or increased levels of proteins such as enzymes, receptors and other cell surface molecules, or other markers of significance (e.g., neurotransmitters, amino acids, neuropeptides and biogenic amines) can be analyzed with any technique known in the art which can identify the alteration of the level of such molecules. These techniques include immunohistochemistry using antibodies against such molecules, or biochemical analysis. Such biochemical analysis includes protein assays, enzymatic assays, receptor binding assays, enzyme-linked immunosorbent assays (ELISA), electrophoretic analysis, analysis with high-performance liquid chromatography (HPLC), Western blots, and radioimmune assays (RIA). Nucleic acid analysis such as Northern blots can be used to examine the levels of mRNA coding for these molecules, or for enzymes which synthesize these molecules.

### D. Preservation of Cells

Once isolated and/or purified, it is sometimes desirable to preserve the HSCs of the disclosure. For example, HSCs can be preserved by freezing in the presence of a cryoprotectant, i.e., an agent that reduces or prevents damage to cells upon freezing. Cryoprotectants include sugars (e.g., glucose or trehalose), glycols such as glycerol (e.g., 5-20% v/v in culture media), ethylene glycol, and propylene glycol, dextran, and dimethyl sulfoxide (DMSO) (e.g., 5-15% in culture media). Appropriate freezing conditions (e.g., 1-3° C. per minute) and storage conditions (e.g., between −140 and −180° C. or at −196° C., such as in liquid nitrogen) can be determined by one of skill in the art. Other preservation methods are described in U.S. Pat. Nos. 5,004,681, 5,192,553, 5,656,498, 5,955,257, and 6,461,645. Methods for banking stem cells are described, for example, in U.S. Patent Application Publication No. 2003/0215942.

### IV. Definitions

To aid in understanding the detailed description of the compositions and methods according to the disclosure, a few express defmitions are provided to facilitate an unambiguous disclosure of the various aspects of the disclosure.

Unless otherwise defined, all technical and scientific terms used herein have the same meaning as commonly understood by one of ordinary skill in the art to which this disclosure belongs.

### ABBREVIATIONS

GGF GATA2, GFI1B, and FOS

3GF GATA2, GFI1, GFI1B, and FOS

GATA2 GATA binding protein 2

FOS FBJ osteosarcoma oncogene or c-Fos

GFI1B Growth factor independent 1B

GFI1 Growth factor independent 1

TFs Transcription factors

ACE Angiotensin-converting enzyme

ACLDL Acetylated low-density lipoprotein

BM Bone marrow

CB Cord blood

CFU Colony forming unit

CFU-GM Colony forming unit-Granulocyte/Monocyte

DA Dorsal aorta

DNA Deoxyribonucleic acid

Dox Doxycycline

EHT Endothelial to hematopoietic transition

FACS Fluorescence-activated cell sorting

FBS Fetal bovine serum

FL Fetal liver

GAG Glycosaminoglycan

G-CSF Granulocyte-colony stimulating factor

GFP Green fluorescent protein

GO Gene ontology

HDFs Human dermal fibroblasts

HE Hemogenic endothelium

hESCs Human embryonic stem cells

HLA Human leukocyte antigen

HSC Hematopoietic stem cell

HSCT Hematopoietic stem cell transplantation

iPSC Induced pluripotent stem cell

LDA Limiting dilution analysis

LTC Long-term culture

LTC-IC Long-term culture-initiating cell

PCA Principal component analysis

PS34 Prom1+Sca1+CD34+CD45−

PSCs Pluripotent stem cells

P-Sp Para-aortic-splanchnopleura

RNA Ribonucleic acid

RTK Receptor tyrosine kinase

RUNX1 Runt-related transcription factor 1

SCL Stem cell leukemia

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

The term “gene” refers to a DNA sequence that encodes a sequence of amino acids which comprise all or part of one or more polypeptides, proteins or enzymes, and may or may not include introns, and regulatory DNA sequences, such as promoter or enhancer sequences, 5′-untranslated region, or 3′-untranslated region which affect, for example, the conditions under which the gene is expressed.

The term “coding sequence” is defined herein as a nucleic acid sequence that is transcribed into mRNA, which is translated into a polypeptide when placed under the control of the appropriate control sequences. The boundaries of the coding sequence are generally determined by the ATG start codon, which is normally the start of the open reading frame at the 5′ end of the mRNA and a transcription terminator sequence located just downstream of the open reading frame at the 3′ end of the mRNA. A coding sequence can include, but is not limited to, genomic DNA, cDNA, semisynthetic, synthetic, and recombinant nucleic acid sequences. In one aspect, a promoter DNA sequence is defined by being the DNA sequence located upstream of a coding sequence associated thereto and by being capable of controlling the expression of this coding sequence.

“Nucleic acid” or “nucleic acid sequence” or “nucleic acid molecule” refers to deoxyribonucleotides or ribonucleotides and polymers thereof in either single- or double-stranded form. The term nucleic acid is used interchangeably with gene, complementary DNA (cDNA), messenger RNA (mRNA), oligonucleotide, and polynucleotide. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). The terms encompass molecules formed from any of the known base analogs of DNA and RNA such as, but not limited to 4-acetylcytosine, 8-hydroxy-N6-methyladenine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3 -methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions, in some aspects, are achieved by generating sequences in which the third position of one or more selected (or all) codons is substituted with mixed-base and/or deoxyinosine residues (Batzer et al., Nucleic Acid Res. 19: 5081, 1991; Ohtsuka et al., J. Biol. Chem. 260: 2605-8, 1985; Rossolini et al., Mol. Cell. Probes 8: 91-8, 1994). The term nucleic acid is used interchangeably with gene, cDNA, mRNA, oligonucleotide, and polynucleotide.

The terms “protein,” “polypeptide,” and “peptide” are used interchangeably herein to refer to a polymer of amino acid residues linked via peptide bonds. The term “protein” typically refers to large polypeptides. The term “peptide” typically refers to short polypeptides.

The terms “identical” or percent “identity” as known in the art refers to a relationship between the sequences of two or more polypeptide molecules or two or more nucleic acid molecules, as determined by comparing the sequences. In the art, “identity” also means the degree of sequence relatedness between nucleic acid molecules or polypeptides, as the case may be, as determined by the match between strings of two or more nucleotide or two or more amino acid sequences. “Identity” measures the percent of identical matches between the smaller of two or more sequences with gap alignments (if any) addressed by a particular mathematical model or computer program (i.e., “algorithms”). “Substantial identity” refers to sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity over a specified sequence. In some aspects, the identity exists over a region that is at least about 50-100 amino acids or nucleotides in length. In other aspects, the identity exists over a region that is at least about 100-200 amino acids or nucleotides in length. In other aspects, the identity exists over a region that is at least about 200-500 amino acids or nucleotides in length. In certain aspects, percent sequence identity is determined using a computer program selected from the group consisting of GAP, BLASTP, BLASTN, FASTA, BLASTA, BLASTX, BestFit, and the Smith-Waterman algorithm.

The term “similarity” is a related concept but, in contrast to “identity,” refers to a measure of similarity which includes both identical matches and conservative substitution matches. If two polypeptide sequences have, for example, 10/20 identical amino acids, and the remainder are all non-conservative substitutions, then the percent identity and similarity would both be 50%. If, in the same example, there are five more positions where there are conservative substitutions, then the percent identity remains 50%, but the percent similarity would be 75% (15/20). Therefore, in cases where there are conservative substitutions, the degree of percent similarity between two polypeptides will be higher than the percent identity between those two polypeptides.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as about 1% to 50%, it is intended that values such as 2% to 40%, 10% to 30%, or 1% to 3%, etc., are expressly enumerated in this specification. The values listed above are only examples of what is specifically intended.

Ranges, in various aspects, are expressed herein as from “about” or “approximately” one particular value and/or to “about” or “approximately” another particular value. When values are expressed as approximations, by use of the antecedent “about,” it will be understood that some amount of variation is included in the range.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all.

As used herein “selectable marker” refers to a gene encoding an enzyme or other protein that confers upon the cell or organism in which it is expressed an identifiable phenotypic change such as enzymatic activity, fluorescence, or resistance to a drug, antibiotic or other agents. A “heterologous selectable marker” refers to a selectable marker gene that has been inserted into the genome of an animal in which it would not normally be found. In some aspects, a selectable marker is GFP or mCherry. The worker of ordinary skill in the art will understand which selectable marker known in the art is useful in the methods described herein.

The term “vector” is used to refer to any molecule (e.g., nucleic acid, plasmid or virus) used to transfer coding information to a host cell. A “cloning vector” is a small piece of DNA into which a foreign DNA fragment can be inserted. The insertion of the fragment into the cloning vector is carried out by treating the vehicle and the foreign DNA with the same restriction enzyme, then ligating the fragments together. There are many types of cloning vectors, and all types of cloning vectors are included for use in the disclosure. An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. In certain aspects, the expression vector includes a nucleic acid to be transcribed operably linked to a promoter.

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “transduction” as used herein refers to the acquisition and transfer of eukaryotic cellular sequences by retroviruses or lentiviruses. The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52:456 (1973); Sambrook et al., Molecular Cloning, a Laboratory Manual, Cold Spring Harbor Laboratories, N.Y., (1989); Davis et al., Basic Methods in Molecular Biology, Elsevier, (1986); and Chu et al., Gene, 13:197 (1981). Such techniques can be used to introduce one or more exogenous DNA moieties into suitable host cells.

The term “introducing” as used herein refers to the transduction or transfection of exogenous DNA into the cell for subsequent expression of the encoded polypeptide in the cell. In some aspects, the methods of the disclosure include introducing a combination of transcription factors into a differentiated cell.

The term “transformation” as used herein refers to a change in a cell's genetic characteristics, and a cell has been transformed when it has been modified to contain new DNA. For example, a cell is transformed where it is genetically modified from its native state. Following transfection or transduction, the transforming DNA may recombine with that of the cell by physically integrating into a chromosome of the cell. In some instances, the DNA is maintained transiently as an episomal element without being replicated, or it replicates independently as a plasmid. A cell is considered to have been stably transformed or transduced when the DNA is replicated with the division of the cell.

As used herein, the term “differentiation” refers to the developmental process of lineage commitment. A “lineage” refers to a pathway of cellular development, in which precursor or “progenitor” cells undergo progressive physiological changes to become a specified cell type having a characteristic function (e.g., nerve cell, muscle cell, or endothelial cell). Differentiation occurs in stages, whereby cells gradually become more specified until they reach full maturity, which is also referred to as “terminal differentiation.” A “differentiated cell,” as used herein, is a cell that has matured so that it has become specialized, i.e., lost its capacity to develop into any specialized cell type found in the body.

As used herein, a “stem cell” is a multipotent, pluripotent, or totipotent cell that is capable of self-renewal and can give rise to more than one type of cell through asymmetric cell division. The term “self-renewal” as used herein, refers to the process by which a stem cell divides to generate one (asymmetric division) or two (symmetric division) daughter cells having development potential indistinguishable from the mother cell. Self-renewal involves both proliferation and the maintenance of an undifferentiated state. Of all stem cell types, autologous harvesting involves the least risk. By definition, autologous cells are obtained from one's own body, just as one may bank his or her own blood for elective surgical procedures. Heterologous cells, therefore, are cells obtained from another source, not from one's own body.

“Totipotent (i.e., omnipotent) stem cells” can differentiate into embryonic and extraembryonic cell types. Such cells can construct a complete, viable organism. These cells are produced from the fusion of an egg and sperm cell. Cells produced by the first few divisions of the fertilized egg are also totipotent. “Pluripotent stem cells” are the descendants of totipotent cells and can differentiate into nearly all cells, i.e., cells derived from any of the three germ layers. “Multipotent stem cells” can differentiate into a number of cells, but only those of a closely related family of cells. For example, hematopoietic stem cells are an example of multipotent stem cells, and they can differentiate into any of the many types of blood cells, but they cannot become muscle or nerve cells. “Oligopotent stem cells” can differentiate into only a few cell types within a tissue. For example, a lymphoid stem cell can become a blood cell found in the lymphatic system, e.g., T cell, B cell, or plasma cell, but cannot become a different kind of blood cells, such as a red blood cell or a platelet; and a neural stem cell can only create a subset of neurons in the brain. “Unipotent stem cells” can produce only one cell type, their own, but have the property of self-renewal, which distinguishes them from non-stem cells, e.g., muscle stem cells.

The term “multipotent,” with respect to stem cells of the disclosure, refers to the ability of the stem cells to give rise to cells of multiple lineages. An “HSC” is self-renewing and is a multipotent cell. Thus, HSCs can be transplanted into another individual and then produce new blood cells over a period of time. In some animals, it is also possible to isolate stem cells from a transplanted individual animal, which can themselves be serially transplanted into other individuals, thus demonstrating that the stem cell was able to self-renew.

As used herein, the term “isolated” refers to a stem cell or population of daughter stem cells in a non-naturally occurring state outside of the body (e.g., isolated from the body or a biological sample from the body). In some aspects, the biological sample includes bone marrow, synovial fluid, blood (e.g., peripheral blood), or tissue.

As used herein, the term “purified” as in a “purified cell” refers to a cell that has been separated from the body of a subject but remains in the presence of other cell types also obtained from the body of the subject. By “substantially purified” is meant that the desired cells are enriched by at least 20%, more preferably by at least 50%, even more preferably by at least 75%, and most preferably by at least 90%, or even 95%.

A “population of cells” is a collection of at least ten cells. In various aspects, the population consists of at least twenty cells. In other aspects, the population consists of at least one hundred cells. In further aspects, the population of cells consists of at least one thousand, or even one million cells or more. Because the stem cells of the present disclosure exhibit a capacity for self-renewal, they could potentially be maintained in cell culture indefmitely.

The term “allogeneic,” as used herein, refers to cells of the same species that differ genetically to the cell in comparison.

The term “autologous,” as used herein, refers to cells derived from the same subject.

As used herein, the term “subject” refers to a vertebrate, and in some exemplary aspects, a mammal. Such mammals include, but are not limited to, mammals of the order Rodentia, such as mice and rats, and mammals of the order Lagomorpha, such as rabbits, mammals from the order Carnivora, including Felines (cats) and canines (dogs), mammals from the order Artiodactyla, including bovines (cows) and swines (pigs) or of the order Perissodactyla, including Equines (horses), mammals from the order Primates, Ceboids, or Simoids (monkeys) and of the order Anthropoids (humans and apes). In exemplary aspects, the mammal is a mouse. In more exemplary aspects, the mammal is a human.

The terms “effective amount” and “therapeutically effective amount” each refer to the amount or number of HSCs necessary to elicit a positive response in the subject in need of HSCT or HSC therapy. For example, an effective amount, in some aspects of the disclosure, would be the amount necessary to carry out HSCT in a subject with a disease, disorder, or condition which could benefit from receiving HSCT and elicit a positive effect on the health of the subject.

A “control,” as used herein, can refer to an active, positive, negative or vehicle control. As will be understood by those of skill in the art, controls are used to establish the relevance of experimental results and provide a comparison for the condition being tested.

Each publication, patent application, patent, and other reference cited herein is incorporated by reference in its entirety to the extent that it is not inconsistent with the present disclosure.

Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

Recitation of ranges of values herein are merely intended to serve as a shorthand method for referring individually to each separate value falling within the range and each endpoint unless otherwise indicated herein, and each separate value and endpoint is incorporated into the specification as if it were individually recited herein.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The section headings as used herein are for organizational purposes only and are not to be construed as limiting the subject matter described.

It is understood that the examples and embodiments described herein are for illustrative purposes only and that various modifications or changes in light thereof will be suggested to persons skilled in the art and are to be included within the spirit and purview of this application and scope of the appended claims.

### V. EXAMPLES

### Example 1

This example describes the materials and methods to be used in the subsequent examples.

### Human Dermal Fibroblast, AFT024, and 293T Cell Culture

Human adult dermal fibroblasts (HDFs, ScienCell) used for all these experiments were obtained from ScienCell. Cells were plated in 10 cm tissue culture dishes in D10 media (Dulbecco's Modified Eagle Medium; Thermo Fisher Scientific) containing 10% fetal bovine serum (FBS; Benchmark), 1 mM L-Glutamine and penicillin/streptomycin (P/S) (10 μg/ml; Thermo Fisher Scientific). 293T cells for viral production were also cultured in standard D10 media at 37° C. AFT024 cells used for LTC and LDA experiments were cultured in D10 media at 32° C. for expansion supplemented with 50 μM 2-ME. The day prior to being used for experiments, AFT024 were mitotically inactivated via irradiation as previously described (Moore, K.A. et al. (1997) Blood 89, 4337-4347), and placed in 37° C.

### Molecular Cloning, Lentivirus Production, and tdT-HDF Generation

The coding regions of every candidate TF were individually cloned into the pFUW-TetO vector where expression is controlled by the minimal CMV promoter and the tetracycline operator. Lentiviral vectors carrying each of the chosen reprogramming factors were generated by calcium phosphate transfection into the 293T packaging cell line with a mixture of the viral plasmids of choice and the constructs that instruct viral packaging and the VSV-G protein (pMD2.G and psPAX2). For activation of the transgenes, lentiviral vectors containing the reverse tetracycline transactivator M2rtTA were co-transduced. M2rtTA is controlled by the constitutively active human ubiquitin C promoter. In this “Tetracycline On” system, after additional of DOX at a concentration of 1 μg/ml in the reprogramming media, the rtTA protein can activate the tetracycline response element (TRE) promoter that will then drive the transcription of the genes of interest. After 293T transfections, the viral supernatant was collected after 36, 48, and 60 hours and then filtered (0.45 μm). Lentivirus carrying the pSin-tdTomato vector (constitutively driven by the EF2 promoter) was generated as described above and used to transduce low passage HDFs. The top 10% of tdTomato⁺ (tdT⁺) cells were sorted and cultured to establish the tdT-HDF line in D10 media.

### Viral Transduction and Reprogrammed Cell Culture

For the majority of these experiments, HDFs were transduced with a viral cocktail consisting of 33.33% D10 media, 33.33% viral supernatant containing M2rtTA, and the remaining 33.33% containing equal portions of each factor within the GATA2, GFI1B, and FOS (GGF) or GATA2, GFI1, GFI1B, and FOS (3GF) TF sets to ensure equal multiplicities of infection of each individual viral particle as well as 8 μg/ml of Polybrene. Control transductions with mOrange in pFUW resulted in >95% efficiency. HDFs on Day −1 were plated at a density of 1.5×10⁵−3.0×10⁵ on 0.1% gelatin-coated 10 cm dishes or across the wells in gelatin-coated 6-well plates with D10 media. On the morning of Day 0, the cells were transduced with the aforementioned viral cocktails. The cells were transduced 2 more times, once on the evening of Day 0 and the morning of Day 1. On the evening of Day 1, media was switched to D10 supplemented with 1 μg/ml DOX to begin transgene activation. On Day 4 transduced HDFs were dissociated with trypLE Express (Thermo Fisher Scientific) and split 1:2 onto 0.1% gelatin-coated plates with Myelocult media (H5100; Stem Cell Technologies) supplemented with hydrocortisone (HC) (10⁻⁶M; Stem Cell Technologies), the cytokines SCF, FLT3L, and TPO (all R&D systems, 25 ng/ml as previously described (Magnusson, M., et al. (2013). PloS one 8, e53912)), and 1 μg/ml DOX. Myelocult media was changed every 4 days for the duration of the cultures.

### FACS Analysis and Sorting

For FACS analysis cells from standard reprogramming, CFU, or LTC experiments were first harvested using trypLE express at specified day points and washed with PBS supplemented with 5% FBS and 1mM EDTA. Flow cytometric analysis was performed on a 5-laser LSRII with Diva software (BD Biosciences) and analyzed with FCS Express 6 Flow Research Edition (Win64). Cells were stained with PE/CY7-hCD45 (2D1), FITC-hCD235a (GA-R2), APC-hCD41 (MReg30), BV421-hCD14 (M5E2), BV421-hCD34 (581), APC-hCD45 or FITC-hCD45 (2D1), PE-hEPCR (RCR-401), or APC-hCD49f (GoH3) (all Biolegend), as well as PE-hACE (BB9), FITC-hCD90 (5E10), PE-hCD49f (GoH3) (BD Biosciences), APC-hCD90 (5E10, Affymatrix Inc.), or hACE-Biotin (BB9, R&D Scientific Corporation) with APC-Cy7 Streptavidin (BioLegend). 4,6-diamidino-2-phenylindole (DAPI, 1 μg/mL, Sigma) or Propidium Iodide (PI, R17755, Invitrogen) was added prior to analysis to exclude dead cells. Sorting for transplants, LTC, and CFU assays were performed with APC-CD49f alone, PE-CD49f alone or BV421-CD34 and PE-CD49f using DAPI or PI to exclude dead cells.

### Flow Cytometry Analysis and Fluorescence-Activated Cell Sorting

Cell cultures were dissociated with TrypLE Express or Accutase Cell detachment solution (Innovative Cell Technologies, Inc) and stained with fluorochrome-coupled antibodies (Key Resource Table). Cell populations were isolated on an InFlux cell sorter (BD Biosciences) and immediately lysed in Trizol (Ambion) for RNA extraction, cultured on 0.1% gelatin-coated 6-well plates in Myelocult media or transplanted. Flow cytometric analysis was performed on a 5-laser LSRII with Diva software (BD Biosciences) and further analyzed using FlowJo software. DAPI (1 μgml⁻¹) was added before analysis to exclude dead cells.

### Coimmunoprecipitation (Co-IP)

Nuclear extracts were prepared from HDFs with ectopic expression of 3×FLAG-tagged GATA2, HA-tagged GFI1B, and FOS and incubated with 5 μg of each antibody (Key Resource Table) The immune complexes were then washed four times with the lysis buffer by centrifugation. IP/co-IP were performed using 5% of input samples. For the control IP, 5 μg of rabbit IgG (Key Resource Table) was used. Samples were heated in SDS sample buffer and processed by western blotting.

### Western Blot Analysis

Cells were lysed in RIPA-B buffer (20 mM Na2HPO4 [pH 7.4], 150 mM NaCl, 1% Triton X-100) in the presence of protease inhibitors (3 μg/mlaprotinin, 750 μg/ml benzamidine, 1 mM phenylmethylsulfonyl fluoride, 5 mM NaF and 2 mM sodium orthovanadate) and incubated on ice for 30 min with occasional vortexing. Samples were centrifuged to remove cell debris and heated in SDS sample buffer. For immunoblotting, membranes were blocked with TBST buffer (10 mM Tris-HCl (pH 7.9), 150 mM NaCl, and 0.05% Tween 20) containing 3% milk, incubated with primary antibodies, washed three times with TBST, incubated with HRP-conjugated secondary antibodies, washed three times with TBST and subsequently detected by ECL or Femto (Thermo Scientific).

### Chromatin Immunoprecipitation (ChIP)-seq

ChIP assays were performed in HDFs transduced with a pool of 3xFLAG-tagged-GATA2, HA-tagged-GFI1B and FOS and the transgenes were induced with Doxycycline. After 48 hr, 20-50×10′6 cells were used for each experiment, and crosslinking conditions were optimized for each factor. For GATA2 and GFI1B ChIP cells were fixed with 11% formaldehyde (Sigma) at room temperature on a rotating platform for 10 min. Formaldehyde was quenched by adding of 125 nM of glycine on a rotating platform for 5 min at room temperature, and cross-linked cells were washed twice in ice-cold PBS. Chromatin shearing was done using the E210 Covaris to a 150-350 bp range, insoluble debris was centrifuged, then sheared chromatin fragments were incubated overnight at 4° C. with antibodies coupled to 50 μl Protein G dynabeads (Invitrogen). For FOS ChIP 3 μg of antibody was used per 5-10×10′6 cells and for FLAG and HA 10 μg of antibody per 20-50×10⁶ cells. Beads were washed five times with RIPA buffer and once with TE containing 50 mM NaCl, and complexes eluted from beads in elution buffer by heating at 65° C. and shaking in a Thermomixer. Reverse cross-linking was performed overnight at 65° C. Whole cell extract DNA was treated for cross-link reversal. Immunoprecipitated and whole cell extract DNA were treated with RNaseA, proteinase K and purified using Phenol:Chloroform: Isoamyl Alcohol extraction followed by ethanol precipitation. For FOS ChIP, 5-10×10⁶ cells were double crosslinked. First, cells were crosslinked in PBS supplemented with Di(N-succinimidyl) glutarate (DSG, ThermoFisher Scientific 20593) at a final concentration of 2 mM for 45 min at room temperature on a rotating platform. After 3 washes in PBS, formaldehyde crosslinking of proteins and DNA was done for 10 min at room temperature at a concentration of 11% formaldehyde (Sigma) in PBS. Formaldehyde was quenched by adding of 125 nM of glycine on a rotating platform for 5 min at room temperature, and crosslinked cells were washed twice in ice-cold PBS. Libraries were prepared using either KAPA Hyper Prep Kit or NEBNext ChIP-seq Library Prep Master Mix Set for Illumina according to the manufacturer's guidelines. Libraries were size-selected on a 2% agarose gel for 200-400 bp fragments and were sequenced on Illumina HiSeq 2000.

### ChIP-seq Data Visualization

To produce the heat maps, each feature (such as peaks of a transcription factor, histone marks) was aligned at GATA2 or GFI1B summits and tiled the flanking up-and downstream regions within ±4 kb in 100 bp bins. To control for input in the data, an input-normalized value as log₂(RPKMTreat)−log 2(RPKMInput) at each bin was computed, where RPKMTreat is RPKM of the corresponding TF or histone and RPKMInput is RPKM of the corresponding whole genome ‘Input.’ The density of DNase-seq signal within ±1 kb around the center of GATA2 or GFI1B summits was plotted and compared it to the resistant sites, which were resized to be in the same range as GATA2 or GFI1B summits.

### Live imaging

Reprogrammed GGF and 3GF cells were taken at Day 14, 20, 28, and 35, placed in 300 μl of 1×PBS with 5% FBS and incubated with PE-hEPCR 1:20 for 15 minutes at 37° C. The antibody mix was then aspirated, cells were washed with 1×PBS with 5% FBS, had their supplemented myelocult media replenished, and were subsequently imaged on a Leica DMI 4000 B using Leica LAS AF software. For CFU colony live stains, the colonies were collected and washed with 1×PBS with 5% FBS. They were then resuspended in 200 μl trypLE express, incubated at 37° C. for 5 minutes, triturated, and washed again with 1×PBS with 5% FBS. Cells were then resuspended in 200 μl of 1×PBS with 5% FBS, loaded into TC treated, sterile μ-Slide VI 0.4 ibiTreat chamber slides (Ibidi, #80606) and imaged on a Leica DMI6000 Inverted scope using Leica LAS AF software.

### 1° CFU, LTC-IC and Cobblestone Area Forming Cell Assays

For initial CFU assays, reprogrammed tdT-HDFs were harvested with trypLE at Day 15, 20, and 25 of reprogramming, washed in 1×PBS, resuspended in 500 ul of DMEM, and dispersed in 3 ml of methylcellulose (h4435, Stem Cell Technologies). The cell suspension was then drawn into an 18G needle with a 5 ml syringe and plated lml per well of a non-TC treated 6-well plate (Costar, #3736). Empty spaces between the wells were filled with sterile H₂O, and the plates were then incubated at 37° C. in 5% CO₂. For LTC assays, 12-well plates were first coated with 0.1% gelatin for at least 30 minutes in 37° C. Expanded AFT024 stromal cells were harvested and seeded at 3×10⁵−3.5×10⁵ cells/ml in D10 media supplemented with 50 μM 2-ME. 1 ml of the cell suspension was plated in each well of the gelatinized 12-well plates. Cells were then grown overnight at 32° C. with 5% CO2. The next day cells were irradiated with 20 Gy. 20-30k Day 15CD49f⁺ sorted cells were then placed in each well with 4 ml of supplemented Myelocult media (with previously described concentrations of HC, SCF, FLT3L, TPO, and DOX according to treatment time points discussed in the experiment) with 50 μM 2-ME and incubated at 37° C. in 5% CO₂. Plates were then observed for colony growth and morphology, with weekly half-media changes for up to 5 weeks. For 1° CFU assays using Lin⁻CD34⁺ CB HSCs, 250 cells were plated in 1 ml of h4435 methylcellulose, observed over 2 weeks, and colony types/total colony numbers were counted. 2,000 Lin⁻CD34⁺ CB HSCs were plated per well of a 12-well plate for LTC cultures as well.

### Colony Imaging, LTC-IC CFU, and Cytospins

Selected wells from the LTC assay for reprogrammed cells as well as Lin⁻CD34⁺ CB HSCs were harvested and plated in CFU assays as previously described. For LTC-IC CFU plating for CB HSCs from LTC assays, 1 well from these cultures was taken and separated into 90% (therefore representative of 1,800 of the initially seeded Lin⁻CD34⁺ CB HSCs) and 10% (200 initial cells) samples. Cobblestone-like and CFU colonies were imaged on a Leica DMI 4000 B automated inverted scope using Leica LAS AF software. After colony derivation in CFU from the LTC assays, colonies were collected in 1×PBS supplemented with 5% FBS, washed, and resuspended in 200 ul trypLE. Colonies were then incubated in trypLE in 37° C. for 5 minutes, triturated to make single cell suspensions, washed, resuspended in 200 μl 1×PBS with 5% FBS, and loaded into cytospin prepared slides. Samples were spun at 250 rpm for 3 minutes, stained with Hematoxylin and Eosin (H&E), and then imaged on a Leica DM5500 upright scope using Leica LAS AF software.

### AFT024 in vitro Limiting Dilution Analysis

AFT024 stroma was cultured as previously described and harvested to a concentration of 350,000 cells/ml in D10 supplemented with 50 μM 2-ME. In 0.1% gelatin-coated 96-well plates, 100 μl of this suspension was plated and allowed to grow overnight at 32° C. with 5% CO₂. The cells were then irradiated with 20 Gy the next day. Following irradiation, the media was replaced with 100 μl of fresh supplemented myelocult. On Day 15 of reprogramming, CD49f⁺ tdT-HDF cells reprogrammed with either GGF or 3GF were sorted and seeded in all 12 wells of Row A of the prepared 96-well plate with 20,000 cells per well. Using a multichannel pipet, 100 μl of the 200 μl cell suspension was taken to Row B, mixed with the 100 μl already present in the wells, and then serially diluted down to Row H with the dilutions as follows: Row A: 10,000 cells per well; Row B: 5,000 cells; Row C: 2,500 cells; Row D: 1,250 cells; Row E: 625 cells; Row F: 312.5 cells; Row G: 156.25 cells; Row H: 78.125. The day after seeding, 100 μl of fresh supplemented myelocult was added to each row. Half media changes were performed weekly, and wells with emerging cobblestone-like colonies were counted after 5 weeks of long-term culture. Stem cell frequency was then calculated using Poisson statistics (Moore, K. A. et al. (1997). Blood 89, 4337-4347) and extreme limiting dilution analysis (ELDA). The same process was used for LDA analysis in Lin⁻CD34⁺ CB HSCs, but instead LDA numbers started at 1,000 cells per well in Row A, 500 in Row B, 250 in Row C, 125 in Row D, 62.5 in Row E, 31.25 in Row F, 15.625 in Row G, and 7.8125 in Row H. CB HSCs were also grown in supplemented myelocult media, but without added DOX.

### mRNA, cDNA, and Library Sample Preparation

3GF reprogrammed HDFs were reprogrammed to Day 15 and D25 and subsequently sorted in triplicate for 2 populations at both time points: CD49f+CD34− and CD49f+CD34+. 10⁵ cells for CD49f+CD34- replicates and at least 3×10⁴ cells for CD49f+CD34+ replicates were sorted into 1×PBS with 5% FBS, pelleted, and RNA was subsequently isolated following the NucleoSpin RNA XS extraction kit (Clontech, 740902.50). cDNA was synthesized and amplified using the SMART-seq v4 Ultra Low Input RNA Kit for Sequencing (Takara Bio USA, 634889). Amplified cDNA was then purified using the Agencourt AMPure XP Kit (Beckman Coulter, A63880). The concentration of the derived cDNA was quantified using a Qubit fluorometer. The quality of the derived cDNA samples was determined using the Agilent High Sensitivity DNA Kit (Agilent, 5067-4626) and an Agilent 2100 Bioanalyzer. cDNA libraries were then created using the Nextera XT DNA Library Preparation Kit (Illumina, FC-131-1024) and the Nextera XT Index Kit (Illumina, FC-131-1001) and subsequently sequenced on an Illumina HiSeq 4000 with about 25 M 100-nt reads per sample.

### RNAseq Analysis

Reads were mapped to the human genome using STAR to avoid high mapping error rates, low mapping speed, and read length limitation/mapping biases (Dobin, A. et al. (2013). Bioinformatics 29, 15-21). Reads mapping to annotated genes were counted using featureCounts (Liao, Y. et al. (2014). Bioinformatics 30, 923-930). Read count normalization, and pairwise differential expression analyses between groups of samples were performed using DESeq2, which is used for differential analysis of count data (normalized read counts in this disclosure) with shrinkage estimation for fold changes to improve the accuracy and stability of the results. Gage, a gene set analysis method that can manage datasets with different sample sizes or experimental designs, was used to perform gene set enrichment tests between pairwise groups of samples. Enrichment tests were performed for custom gene sets extracted from prior literature and for gene sets associated with specific gene ontology (GO) terms. Samples were additionally analyzed using principal component analysis (PCA) and hierarchical clustering in R. DESeq2 normalized read counts for all relevant samples were plotted using GraphPad Prism 7 software.

### NSG Mouse Transplants

After sorting CD49f cells from 3GF reprogrammed HDFs cultured on 0.1% gelatin, cells were washed in 1×PBS and then transplanted (3.0×10⁵ cells) into NOD-scid IL2Rg^{null}(NSG) 0-2 day old pups via intrahepatic injection. Mouse PB was analyzed 4, 8, and 16 weeks post-injection for engraftment of human-derived cells. To distinguish levels of engraftment, cells were stained for mouse PacBlue-mCD45 (30-F11, Biolegend) and PE-Cy7-hCD45 (2D1, ebioscience). Within the hCD45 compartment, cells stained with APC-eflour 780-hCD3 (UCHI1), PerCP-Cy5.5-hCD8 (RPA-T8), PE-hCD19 (HIB19) and Alexa488-hCD14 (all from Biolegend) were analyzed. Engraftment levels were compared to levels found from cord blood-derived CD34⁺ hematopoietic progenitors isolated using Diamond CD34 Isolation Kits (Miltenyi) using the same injection method as previously described (1.0×10⁵ cells/pup).

### Statistical Analysis

Data were analyzed with GraphPad Prism 7 software using the nonparametric Mann-Whitney test for samples not assuming a normally distributed data set. Bars represent mean, and error bars represent standard error of the mean (SEM). Statistically significant differences are as follows: *p<0.05, **p<.01, ***p<0.001, and ****p<0.0001.

### Example 2

### Screening of Multiple Hematopoietic TFs Reveals GFI1 as the Causative Factor for Progenitor Expansion in vitro

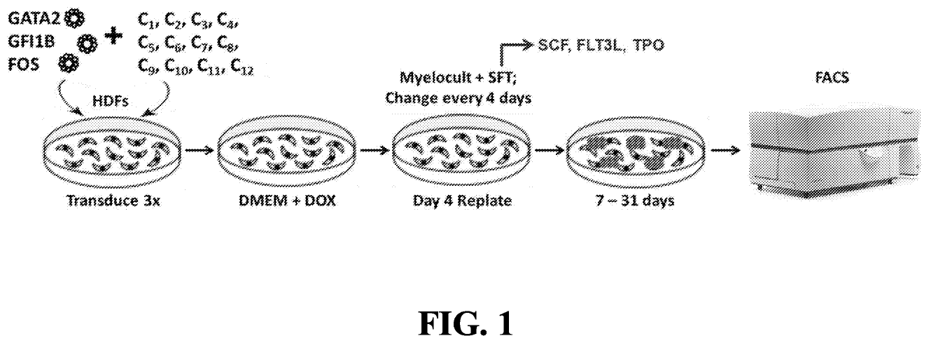

Initial data demonstrated that the three TFs GATA2, GFI1B, and FOS were sufficient to induce a hemogenic program in human fibroblasts (FIG. 1). Although CD49f cells sorted at day 25 of reprogramming could engraft immunocompromised mice, the overall yield of reprogrammed functional cells remains to be improved. To identify a candidate TF to add to the hemogenic induction cocktail in an attempt to increase the yield and functionality of the derived cells, the TFs of various established reprogramming strategies were individually cloned into the doxycycline-inducible pFUW cassette. These TFs were all arranged into 12 distinct combinations that were used in concert with GGF to induce hemogenesis in adult HDFs (FIG. 1).

Through monitoring CD34 induction, each set of reprogrammed cells were screened at day 30 to determine which improved upon the reprogramming efficacy of the GGF cocktail. Via this method, 3 cocktails were found to significantly improve yields of CD34⁺ progenitors. C2 contains the polycistronic STEMCCA pluripotency reprogramming cassette, and C10 contains a combination of shRNAs to p53, which has been shown to improve reprogramming efficiency upon repression of p53. Since the inventors sought to avoid both reprogramming to pluripotency and altering the p53 network of the reprogrammed cells, these cocktails were not used further. C12, however, contained solely a group of TFs: FOSB, GFI1, RUNX1c, and SPI1 (FGRS), which improved GGF reprogramming without induction of pluripotency.

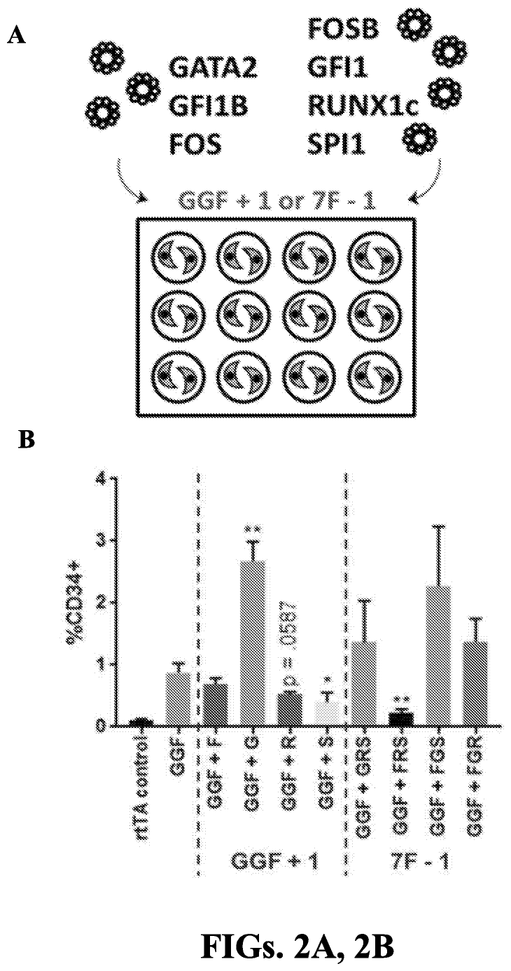

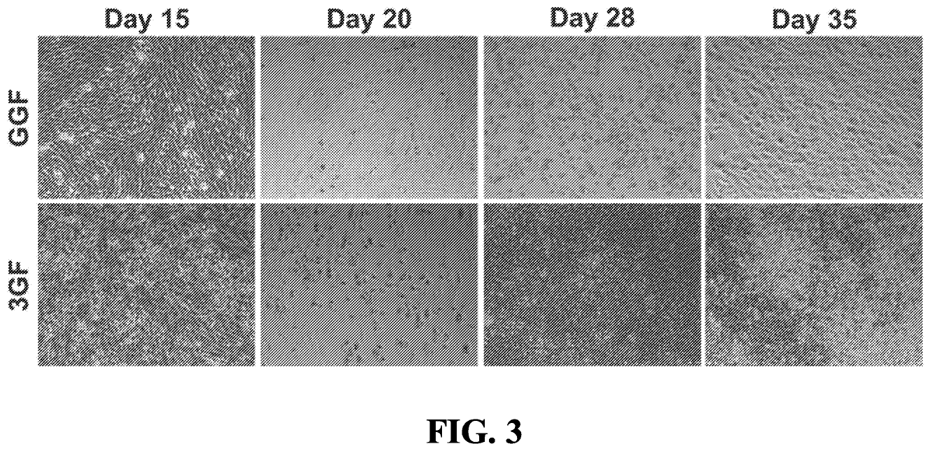

To determine if one or multiple TFs within the FGRS set act with the GGF cocktail, an N−1 or N+1 experiment (FIG. 2A) was carried out. Through this, GFI1 was identified as the factor that improves the yield of CD34⁺ progenitors as seen when GFI1 alone is added to the GGF cocktail, or when GFI1 is removed from the 7-factor cocktail (GGF+FGRS) (FIG. 2B). These experiments conclude that GFI1 added to the GGF cocktail, now termed 3GF (for GATA2, GFI1, GFI1B, and FOS) provides the optimal yield of CD34⁺ progenitors in HDFs. Looking at cell morphology, the changes remain clear between the GGF and 3GF samples, with a large expansion of round hematopoietic-like cells in cells reprogrammed with 3GF (FIG. 3). Because the GGF and FGRS cocktails possess 2 sets of paralogs (GFI1B: GFI1 and FOS: FOSB), the reprogramming cocktails were altered to determine if any factors were interchangeable. Interestingly, removal or substitution of GFI1B greatly increased the yield of CD34⁺ cells, while removal or substitution of FOS completely ablated reprogramming. Additionally, it is clear that GATA2 and FOS are required for hemogenic induction. Previous work demonstrates that GATA2 and FOS reprogramming alone, while expanding the CD34⁺ pool, does not generate CD45⁺ cells with prolonged culture. This demonstrates the necessity of GFI1B for hemogenic induction in HDFs. Using in-house developed software “GPSforGenes,” the inventors found that the TF combinations for GGF and 3GF, as well as GATA2+FOS or GATA2+GFI1+FOS were all highly expressed both in CD34⁺ HSPCs and placental tissue.

### Example 3

### Inclusion of GFI1 to the Reprogramming Cocktail Expands all Hematopoietic Populations of Interest Based on Cell Surface Immunophenotype

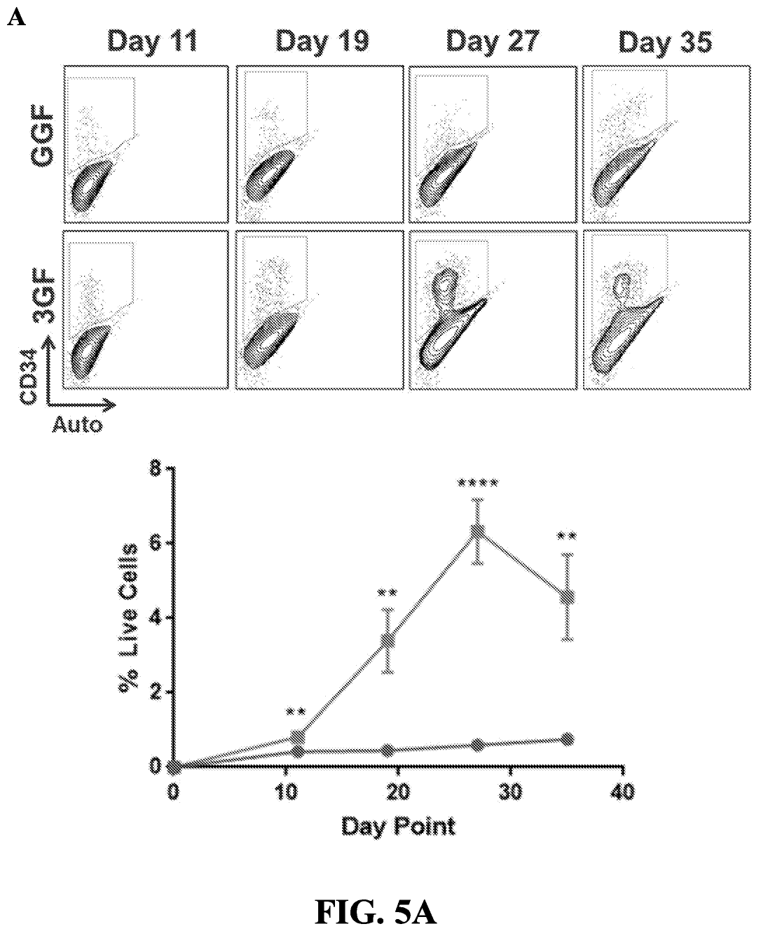

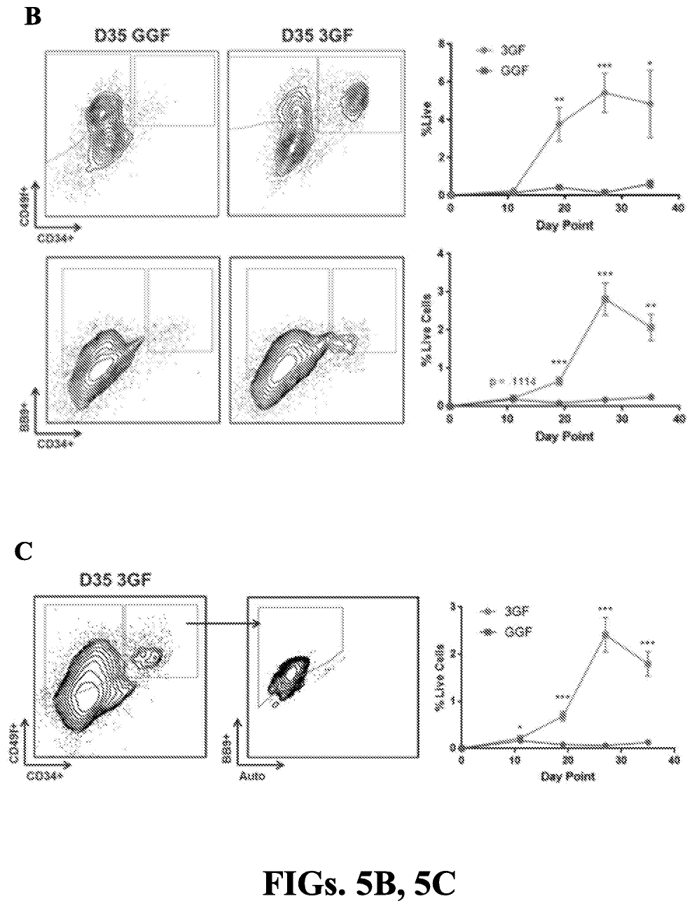

The surface immunophenotype of the 3GF derived cells was then characterized and their yields were compared to those generated from GGF reprogramming. Cell surface marker expression at several time points of the reprogramming for both GGF and 3GF were analyzed. Staining of the 30-day human embryo shows that CD49f and BB9 both mark cells in the mesenchyme of the dorsal aorta, while only BB9 marks the cells residing in the dorsal aorta where active hematopoiesis takes place (FIG. 4A). CD49f, also known as integrin a6, has been shown to enrich for HSCs (Notta, F. et al. (2011). Science 333, 218-221). Likewise, BB9 also enriches for HSCs, and also marks all the cells with a hematopoietic fate in the developing embryo (Jokubaitis, V.J., et al. (2008). Blood 111, 4055-4063; Zambidis, E.T., et al. (2007). Annals of the New York Academy of Sciences 1106, 223-232). With 3GF reprogramming, in the reprogrammed cells, expanded yields of all populations of hematopoietic progenitors (in this case BB9⁺, CD49f, or BB9⁺CD49f⁺ cells) were observed (FIG. 4B). Expanded populations of CD34⁺ throughout reprogramming, as well as CD49f⁺CD34⁺ and BB9⁺CD34⁺ populations with 3GF, were also observed (FIGS. 5A and 5B). Likewise, expansion of CD49f⁺BB9⁺ CD34⁺ cells at each time point of analysis when reprogramming with 3GF was observed (FIG. 4C).

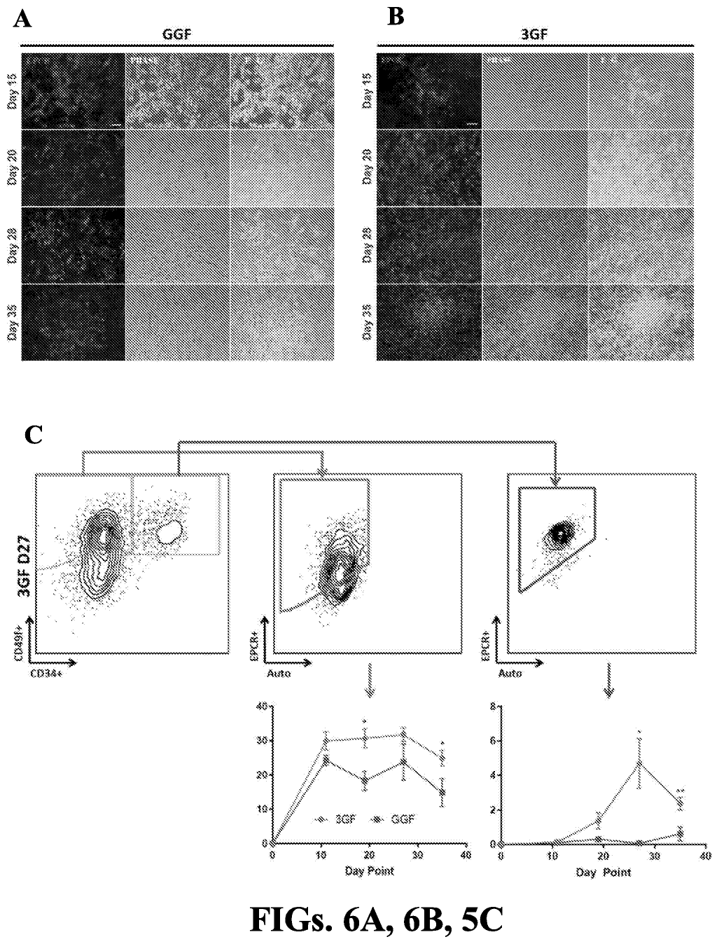

Previous studies have identified EPCR (CD201) as a marker of expanded CD34⁺ progenitors from CB (Fares, I. et al. (2017). Blood 129, 3344-3351) as well as functional HSCs in murine BM (Balazs, A.B., et al. (2006). Blood 107, 2317-2321). Interestingly, GGF and 3GF reprogrammed cell subsets throughout the reprogramming process both stain positive for EPCR. Additionally, the EPCR population expands over time and by morphology appears to marks both endothelial-like cells as well as the rounded HSC-like cells that emerge in these prolonged cultures (FIGS. 6A and 6B). As with the other previous shown HSC markers (FIGS. 5A and 5B), greater numbers of CD49f⁺CD34⁺EPCR⁺ cells were obtained in the 3GF population, with the majority of EPCR⁺cells emerging around day 27, which corresponds to a late-intermediate time point in the reprogramming. While a large subset of CD49f⁺CD34⁻ cells stain positive for EPCR, virtually all of the of CD49f⁺CD34⁺ cells are EPCR⁺, denoting a more purified HSC population (FIG. 6C).

With the addition of GFI1 to the reprogramming cocktail, as well as the cytokines SCF, FLT3L, and TPO to the myelocult media, it was hypothesized that the derived cells would possess unique expression profiles compared to GGF reprogramming while still undergoing a developmental process as cells transition through endothelial intermediates to form hematopoietic cells. To this end, 4 different populations of 3GF reprogrammed cells in triplicate: 1) D15 3GF CD49f⁺CD34⁻; 2) D15 3GF CD49f⁺CD34⁺; 3) D25 3GF CD49f⁺CD34⁻; and 4) D25 3GF CD49f⁺CD34⁺ were sequenced.

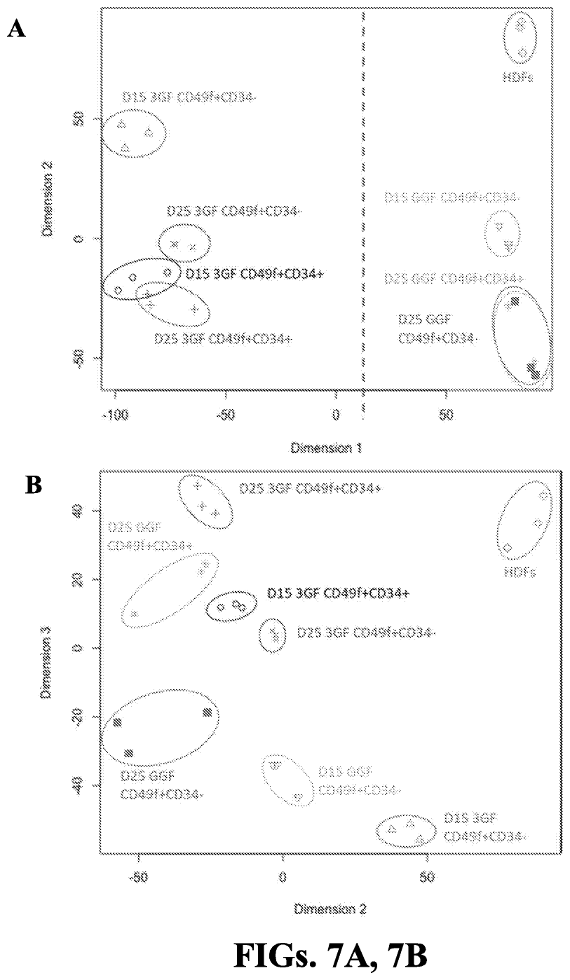

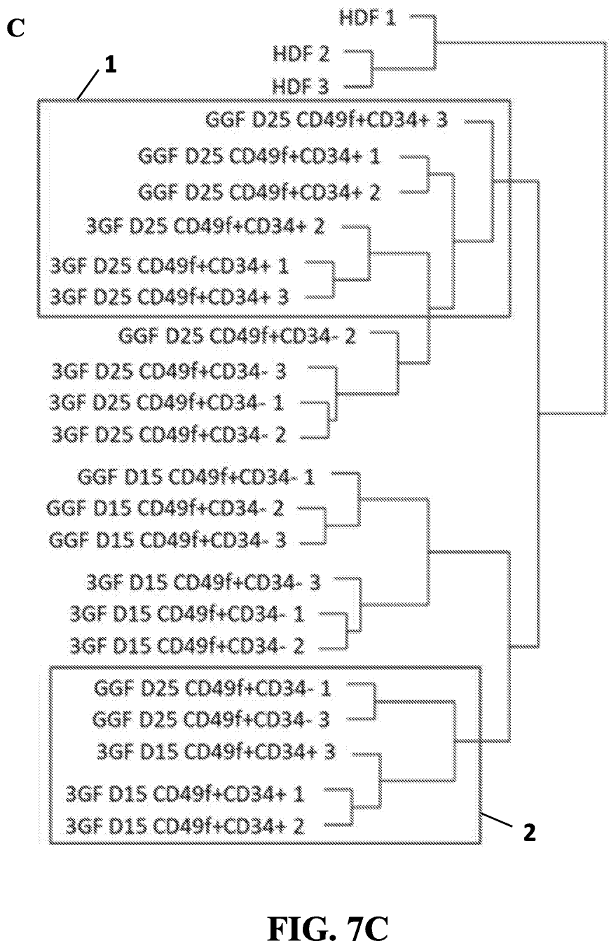

For initial analyses, PCA plots, RNAseq data for HDF negative controls, and data from both D15/D25 CD49f⁺CD34⁻ GGF and D25 CD49f⁺CD34⁺ GGF populations in triplicate were generated. When comparing dimension 1 and 2 between GGF and 3GF cells, what appeared to be batch effects between the 2 datasets were observed (FIG. 7A). Comparison of dimension 2 and 3, however, revealed several other interesting biological differences between GGF and 3GF cells. Notably, specific GGF and 3GF populations clustered similarly but remained distinct. As expected, HDF negative controls clustered completely separate from the populations of interest. Interestingly, the D15 CD49f⁺CD34⁻ populations of both reprogramming sets cluster close together, both distinct from their CD49f⁺CD34⁺ counterparts. It was also observed that the more hematopoietic D25CD49f⁺CD34⁺ GGF cells seem to cluster quite close to the 3GF D15 and 25 CD49f⁺CD34⁺ populations as well as the 3GF D25 CD49f⁺CD34⁻ population. This indicates a more robust acquisition of a hematopoietic fate (FIG. 7B).

After depleting the variation seen in dimension 1, hierarchical clustering analysis first reiterates that HDF negative controls cluster completely separately from all reprogrammed cells. Through this analysis, a close relation of GGF and 3GF D25 CD49f⁺CD34⁺ as well as 3GFD25 CD49f⁺CD34⁻ cells (FIG. 7C, box 1) seems to exist. Additionally, a close relationship between D15 GGF and 3GF CD49f⁺CD34⁻ cells was observed. Interestingly, there was close clustering of the functional population 3GF D15 CD49f⁺CD34⁺ (discussed further in EXAMPLE 5) and GGF D25CD49f⁺CD34⁻, the population that clustered closest to bona fide HSCs in other PCA plots comparing GGF reprogrammed cells and microarray data from CD49f sorted CB HSCs (FIG. 7C, box 2 and data not shown). As did the PCA analysis, this hierarchical clustering demonstrates that while most populations cluster similarly, GGF and 3GF populations remain distinct. Furthermore, the similarities in these two gene expression signatures may offer clues to the key drivers necessary for adopting stem cell function akin to endogenous HSCs.

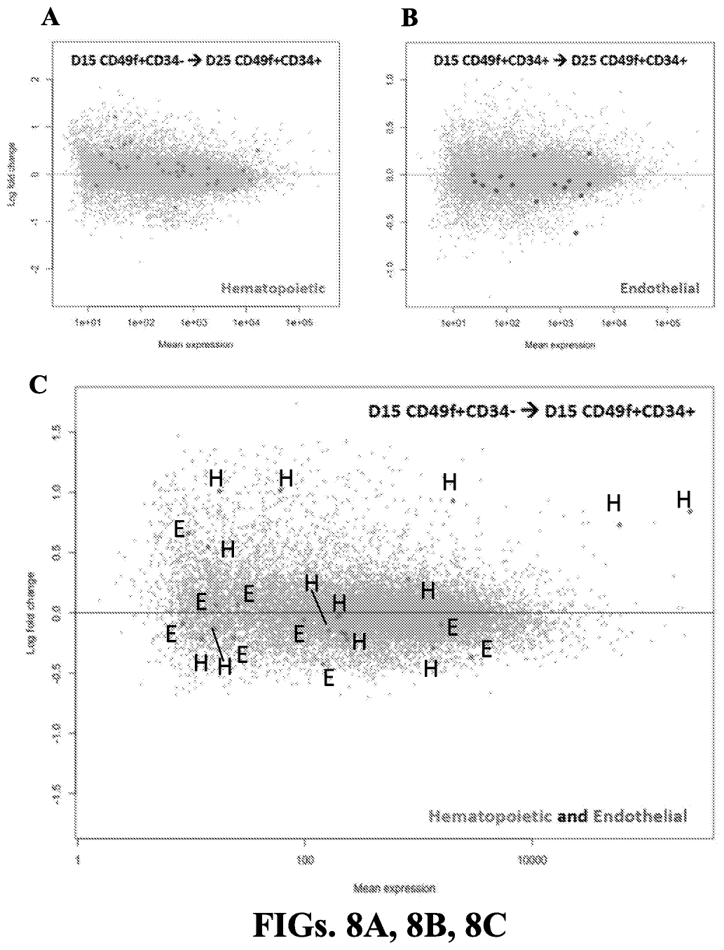

Focusing on 3GF reprogrammed cells, to variety of comparative analyses between the different isolated populations of this dataset were completed. Comparing the D15 CD49⁺CD34⁻ (theorized endothelial intermediates) and D25 CD49f⁺CD34⁺ (theorized hematopoietic cells) datasets, an upregulation of 2273 genes and downregulation of 2965 genes were observed. Using a gene list of upregulated genes in CD49f HSCs as compared to CD90⁻ CD49f MPPs, a statistically significant upregulation of these genes was observed in the 3GF dataset, p<0.05 (FIG. 8A, dots are genes from Notta et al., 2011 (Notta, F. et al. (2011). Science 333, 218-221)). Interestingly, comparative analysis of D15 CD49f⁺CD34⁺ and D25 CD49f⁺CD34⁺ with highlighted endothelial genes shows a significant downregulation of this gene list, p<0.01 (FIG. 8B, dots are endothelial genes). Comparing the uniquely derived D15 CD49f⁺CD34⁺ population as described in this disclosure to D15 CD49⁺CD34⁻ cells, a significant upregulation of an HSC gene list and downregulation of an endothelial gene list were observed, both derived from Guibentif et al., 2017 (Guibentif, C. et al. (2017). Cell reports 19, 10-19) (FIG. 8C, HSC genes, and endothelial genes are indicated--dots next to the letter “H” represent HSC genes and dots next to the letter “E” represent endothelial genes). These comparative analyses highlight and support a developmental trajectory initiated by this reprogramming, as hematopoietic cells emerge from endothelial intermediates as they mature throughout the induction process.

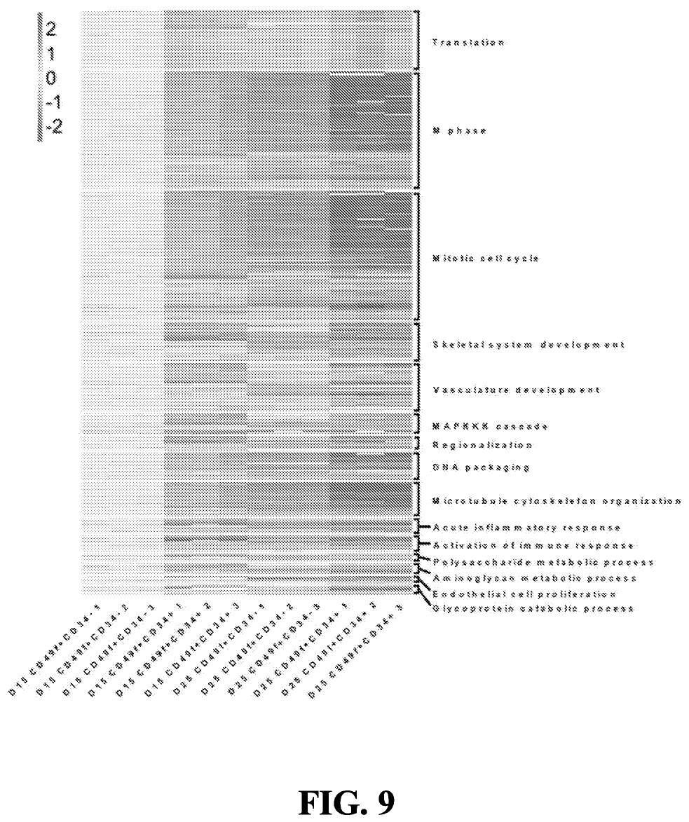

Within the 3GF datasets, to assess globally which pathways were significantly up or downregulated in these cells, the D15 CD49⁺CD34⁻ cells were used as a baseline for subsequent GO term analysis. As a result, the top 15 up or down-regulated terms were observed in other 3 populations as compared to this baseline were identified. A consistent downregulation of key pathways pertaining to the cell cycle, including M phase, mitotic cell cycle, DNA packaging, and microtubule cytoskeleton organization GO terms, from the baseline to the remaining 3 populations (D15 CD49f⁺CD34⁺, D25 CD49f⁺CD34⁻, and D25 CD49f⁺CD34⁺ was also observed (FIG. 9). This suggests that the cells generated later in the reprogramming (as well as the disclosed unique D15 CD49f⁺CD34⁺ cells) shut down the machinery required for the cell cycle, which is consistent with known inactive cell cycle machinery in endogenous quiescent HSCs that usually remain in the GO phase in the BMSeveral studies show that quiescence (or a low proliferation rate) is critical for primitive stem cell maintenance and self-renewal, supporting the notion that in the late stages of reprogramming HSC-like cells characterized by a relatively inactive cell cycle were generated.

This analysis also identified a few key terms that are significantly upregulated from the baseline to the 3 other populations within the 3GF reprogramming dataset. This includes the GO terms skeletal system/vasculature development, acute inflammatory response, activation of the immune response, polysaccharide metabolic process, aminoglycan metabolic process, and glycoprotein catabolic process. Upregulation of the skeletal system/vasculature development terms could indicate enrichment for a response to HSC-support factors typically found in the endogenous HSC niche (Poulos, M.G. et al. (2015). Stem cell reports 5, 881-894). Unsurprisingly, the terms for the inflammatory and immune response indicate activation of hematopoietic-type genes, as HSCs are involved in these pathways (King, K.Y. and Goodell, M. A. (2011). Immunology 11, 685-692). Several terms involved with metabolism and catabolism of aminoglycans, polysaccharides, and glycoproteins (FIG. 9). This directly correlates with the inventors' hypothesis that HSC-like cells generated through this reprogramming work together with stroma that provide the necessary for their maturation (and are therefore primed to process signals from an instructive niche). The AFT024 FL stromal line, in particular, is known to provide a variety of GAGs and other proteoglycans such as DPT, and definitely imparts functional potential on the disclosed reprogrammed cells (discussed in detail in EXAMPLE 5).

After DESeq2 normalization, the induction of key endothelial and hematopoietic genes in the 3GF populations was observed. Among endothelial genes, expression of Von Willebrand Factor (VWF) was observed. VWF is a factor known to be expressed in both endothelial cells and HSCs, with VWF HSCs seemingly capable of multilineage function with a bias towards megakaryocyte and platelet production. Although expression of VWF was found in all of the populations, it was predominately expressed in the CD49f⁺CD34⁺ hematopoietic cells. Similarly, a predominance of ETS2 and FOXC2 in the disclosed CD49f⁺CD34⁻ cells was also observed. These genes both play a role in HE, with ETS2 a target of SCL to control hemato-vascular genes in HE (Org, T., et al. (2015). The EMBO Journal 34, 759-777) and FOXC2 being targeted by NOTCH1 to specify HE in mouse and zebrafish embryos (fang, I. H., et al. (2015). Blood 125, 1418-1426). These both support the idea that the intermediate cells adopt an HE-like fate that can generate the HSC-like cells. Interestingly, downregulation of CXCL5 and ANGPTL4 across time and throughout the populations was also found. CXCL5has been shown to regulate the migration of HSCs from the osteoblastic and endothelial niche (Yoon, K. A., et al. (2012). Stem cells and development 21, 3391-3402) while ANGPTL4 maintains the in vivo repopulation capacity of CD34⁺ human CB cells (Blank, U., et al. (2012). European journal of hematology 89, 198-205). The loss of these genes over time may suggest that the disclosed functional cells can only emerge early in the reprogramming, a notion corroborated by the functional data (see EXAMPLE 5). JAG1, on the other hand, appears to increase as time goes on and as 3GF cells acquire CD34. Studies show that conditional deletion of JAG1 in endothelium results in a severely inhibited hematopoiesis with subsequent exhaustion of the adult HSC pool (Poulos, M. G., et al. (2013). Cell reports 4, 1022-1034) (FIG. 10). This further indicates the endothelial identity the cells take on and show that an intrinsic endothelial niche can be constructed within the disclosed reprogramming.

The induction of key hematopoietic genes throughout the reprogramming, several of which confirm what the results in the flow data was observed (FIGS. 4-6). CD34 expression limited to the CD34⁺ populations validates the accuracy of the sorting and sequencing. Interestingly, although expression of the other markers in all populations was also seen, ACE (BB9), EPCR, and ITGA6 (CD49f) were predominantly expressed in the CD49⁺CD34⁺ populations. These markers are known to purify for functional human HSCs (Notta, F. et al. (2011). Science 333, 218-221; Ramshaw, H. S. et al. (2001). Experimental Hematology 29, 981-992; Jokubaitis, V. J. et al. (2008). Blood 111, 4055-4063; Zambidis, E. T. et al. (2007). Annals of the New York Academy of Sciences 1106, 223-232; Fares, I. et al. (2017). Blood 129, 3344-3351), further confirming that the HSC-like identity was induced in the disclosed 3GF reprogramming.

The upregulation of other hematopoietic genes in the datasets was also observed. Interestingly, there was increased expression of F11R, EPCAM, and CD9 in the D15 CD49f⁺CD34⁺ cells as compared to the D25 CD49f⁺CD34⁺ cells. F11R is highly expressed in the CD34⁺cKit⁺ enriched HSC fraction (Sugano, Y. et al. (2008). Blood 111, 1167-1172) and regulates HSC fate through NOTCH (Kobayashi, I. et al. (2014). Nature 512, 319-323).

EPCAM is expressed in the AGM and associated with CD49f expression (Gomes Fernandes, M. et al. (2018) Molecular human reproduction). CD9 is a tetraspanin protein that plays a key role in CB CD34⁺ HSC migration, adhesion, and homing (Leung, K.T. et al. (2011). Blood 117, 1840-1850). The upregulation of all these major genes further supports the functional data (see EXAMPLE 5) indicating that the 3GF D15 CD49f⁺CD34⁺ population is the one with functional potential, and possibly the disclosed more HSC-like cells from this reprogramming process. Unsurprisingly, RUNX1 expression in all the populations was also found, further supporting the thought that the disclosed cells are hemogenic and theoretically undergoing EHT. Interestingly, HGF has been found to act as a mobilizer of HSCs to the PB through the RTK c-MET (known to be involved with stem cell-mediated liver regeneration (Ishikawa, T. et al. (2012). Hepatology 55, 1215-1226)) without compromising their functional potential. BM stroma produces this HGF, which can work synergistically with G-CSF to induce this observed mobilization (Weimar, I.S. et al. (1998). Experimental Hematology 26, 885-894; Jalili, A., et al. (2010). Stem cells and development 19, 1143-1151). Overall, the data indicate that 3GF reprogramming first induces a HE fate, which then continues on to produce HSC-like cells later on the hemogenic induction process.

### Example 4

### Co-culture of Reprogrammed Cells on AFT024 Monolayers Permits Derivation of Functional Cells Based on Colony forming Potential

TdT-3GF cells reprogrammed to day 15, 20, and 25 showed hematopoietic morphology in reprogramming cultures, but when harvested and plated in CFU assays they do not form colonies. Although the derived 3GF cells clearly displayed a cell surface phenotype highly similar to endogenous human HSCs (FIGS. 4-6), their in vitro functional potential was further optimized by including a separate maturation step in the form of a co-culture system.

To this end, 250 cells per ml of Lin⁻CD34⁺ CB HSCs were plated directly into CFU assays. Discernable CFU-GEMM, BFU-E, and CFU-GM emerged in these cultures after 2 weeks that possessed a variety of hematopoietic cells upon cytospin, demonstrating that these enriched progenitor cells indeed function directly into CFU assays. Quantification of these cells showed an average of 71.33 (SD of 19.442) colonies per 250 initially seeded cells, signifying a colony forming potential of roughly 1 in 3.5 Lin⁻CD34⁺ cells. From these counts, an average of 9.333 CFU-GM (SD of 5.339), 6.44 BFU-E (SD of 2.506), and 55.556 CFU-GEMM (SD of 14.293) were observed. FACS analysis of these colonies revealed that about half of these cells are CD45⁺, and within this population the majority is CD235a⁺CD14⁻. A population of CD235a⁺CD14⁺ cells, as well as CD235a⁻CD14⁺ cells, was also observed. A small population of CD41⁺ cells also emerges from these colonies.

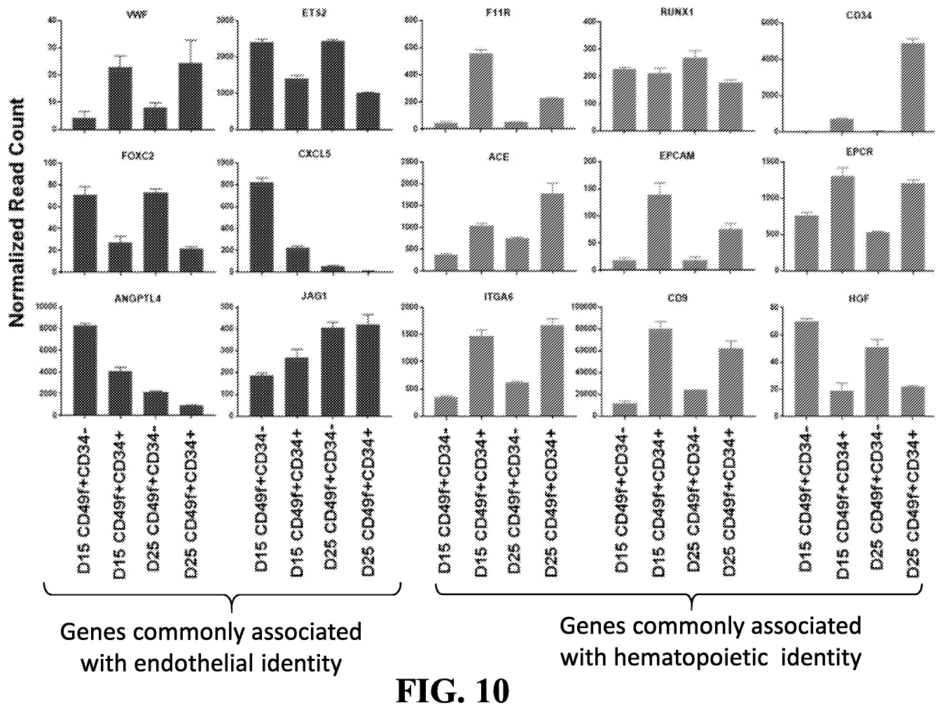

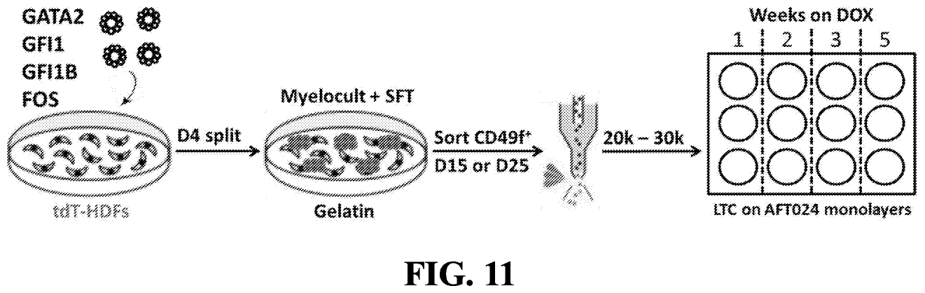

To incorporate an in vitro maturation system into this study, 3GF reprogrammed tdT-HDFs were sorted for CD49f on both day 15 and day 25 of reprogramming and subsequently plated on confluent monolayers of AFT024 with varying lengths of DOX exposure (FIG. 11). Strikingly, when day 15 CD49f sorted cells were cultured on AFT024 for 5 weeks with continuous DOX exposure for the length of the cultures, clear cobblestone-like colonies emerged (FIG. 12A). When these cells were harvested and plated in methylcellulose assays, the formation of hematopoietic colonies with various morphologies upon cytospin was seen (FIG. 12B). These colonies were dissociated and live stained for CD45 and stain positive for various mature lineage markers (FIG. 12C). Cobblestone-like colonies with GGF reprogrammed cells also emerged. However, the quality and quantity of the derived colonies in subsequent CFU assays demonstrate that GGF reprogrammed cells do not possess the expanded functionality observed in 3GF cells after AFT024 co-culture. This establishes AFT024 as a stromal co-culture niche that imparts the signals beneficial for 3GF reprogrammed cells to further mature and adopt hematopoietic functional potential. Additionally, identification of this robust co-culture system allows for several other in vitro experiments to assess the cells derived from the reprogramming.

### Example 5

### LDA of Reprogrammed Cells Reveals Stem Cell Frequency

Using this AFT024 in vitro maturation system, the stem cell frequency of both GGF and 3GF reprogrammed cells can be determined via LDA and Poisson statistics (FIG. 13A). This system allowed for distinct identification of positive cobblestone colonies as compared to tdT-HDFs that did not form colonies (FIG. 13B). Quantification of cobblestone-like colony formation demonstrated a stem cell frequency of 1/4020 in 3GF reprogrammed cells (95% CI=1/2740−1/5899) as compared to 1/7465 (95% CI=1/4834−1/11527) in GGF cells (FIG. 13C). Via this method, a significant difference between stem cell frequencies of these two cell sets was observed, with 3GF reprogramming permitting a significantly greater induction of HSC-like cells.

Experiments using Lin⁻CD34⁺ CB HSCs were performed and the formation of large cobblestone-like colonies were observed after 5 weeks of LTC on AFT024. Cytospin of these cells revealed a majority of myeloid cells as well as some erythroid cells (FIG. 13D). 1° LDA of CB HSCs shows a steady decrease in HSC frequency over 5 weeks, demonstrating that short-term progenitors in these purified cells expand and exhaust, leaving behind true long-term HSCs (FIG. 13E).

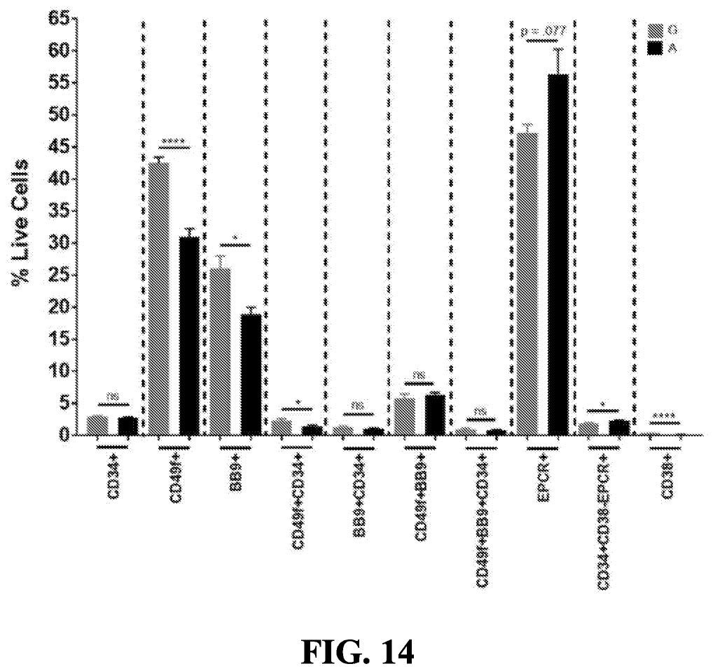

To determine if it is possible to isolate relevant populations from LTC on AFT024, FACS analysis was performed for several of the aforementioned markers on day 15 CD49f⁺cells sorted onto either gelatin or AFT024 coated plates for 5 weeks with DOX supplementation. It was found that though some populations show a significant decrease in cell yield (CD49f⁺, BB9⁺ and CD49f⁺CD34⁺, other populations remain the same (CD34⁺, BB9⁺CD34⁺, CD49f⁺BB9⁺ and CD49f⁺BB9⁺CD34+) or even increase in yield (EPCR⁺ and CD34⁺CD38⁻EPCR⁺. Additionally, the more mature CD38⁺ population significantly decrease in cells grown on AFT024 (FIG. 14). This provides solid evidence that some progenitor populations are maintained (or even expanded) in AFT024 co-cultures, and that these populations can be isolated and sorted for downstream applications.

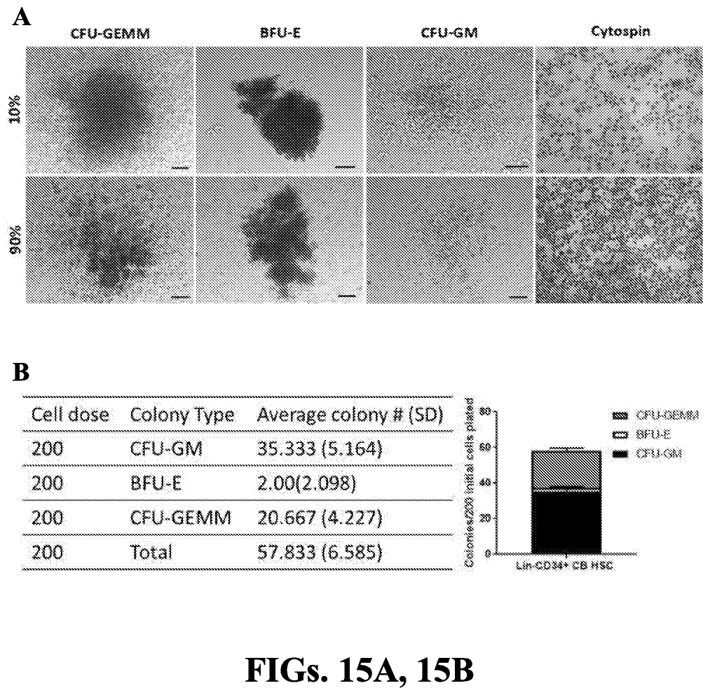

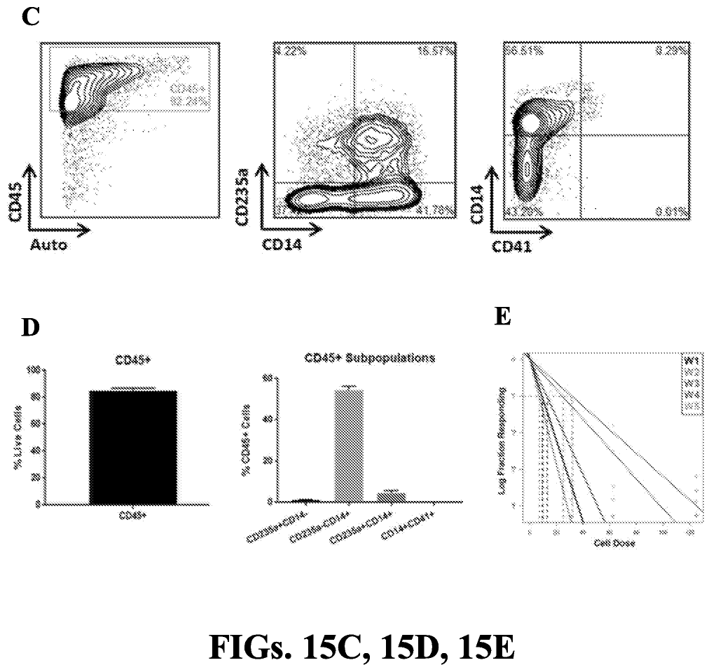

Additional proof of principle experiments using Lin⁻CD34⁺ CB HSCs after AFT024 LTC shows the continued derivation of multilineage colonies in 2° CFU assays after 5 weeks of LTC on AFT024 (FIG. 15A and 15B), with a majority of CD45⁺ cells composed primarily of CD14⁺ myeloid cells (FIGS. 15C and 15D). 2° LDA after 5 weeks of AFT024 co-culture shows a sustained, higher stem cell frequency as compared to 1° LDA assays, signifying the maintenance of true HSCs in vitro with this AFT024 cell line (FIG. 15E).

### Example 6

### Transplantation of Reprogrammed Cells with the Optimized Cocktail Shows Engraftment with Short-Term Multilineage Potential

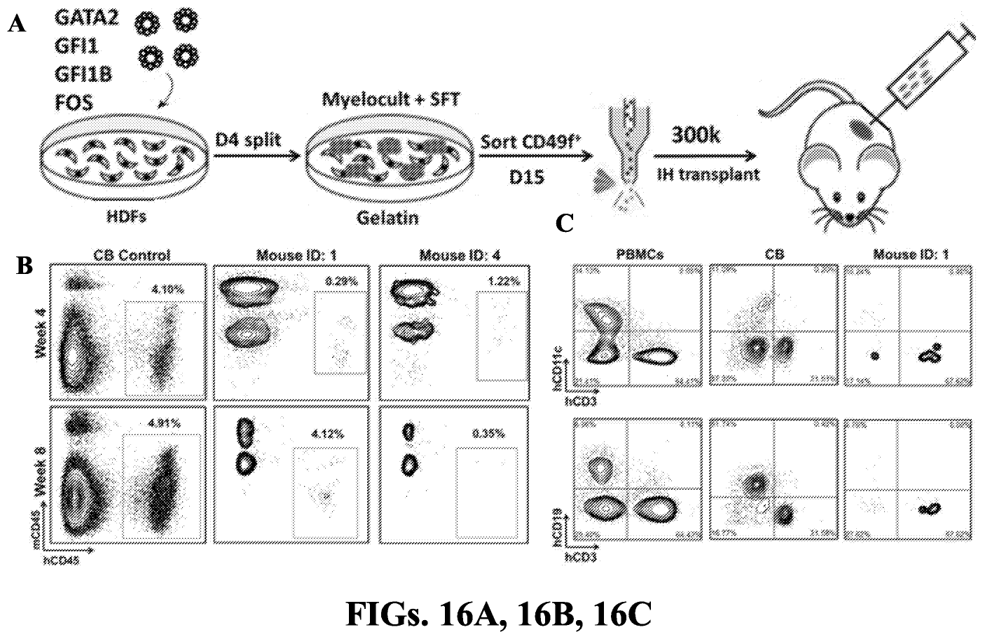

To determine the engraftment potential of 3GF cells, derived CD49f⁺ cells were sorted at three different time points (D12, D15, and D18) of the reprogramming and transplanted into immunocompromised mice (FIG. 16A). It was hypothesized that given the success of D15 CD49f⁺ sorted cells in the in vitro LTC, CFU, and LDA assays, cells sorted at an earlier time point than the inventors' previous work would function superiorly. Indeed, with 3GF sorted cells, cells sorted at D15 could engraft, but not D25 CD49f⁺ sorted cells (FIGS. 16A and 16B).

### Example 7

### GATA2 and GFI1B Interact and Share a Cohort of Target Sites and Engage Open Promoters and Enhancers Regions

This disclosure also provides results related to the extent of overlap between GATA2 and GFI1B genomic targets and their interactions. By displaying GATA2 and GFI1B target sites, it was found that 750 genomic positions were shared, representing 31.6% of total GFI1B targets. These include HSC and EHT regulators such as PRDM1 and PODXL. Motif comparison analysis showed a significant similarity between GATA2 and GFI1B motifs (Jaccard similarity index=0.1) (Vorontsov, I. E., et al. (2013). AMB 8, 23), supporting the interaction between the two TFs. de novo motif prediction for the overlapping peaks was then performed. Interestingly, AP-1 motif was the most enriched followed by the GATA and GFI1 motifs, highlighting the cooperative action among the 3 factors during reprogramming. Co-bound genes are part of pathways such as interferon-gamma signaling, inflammation and cytoskeletal regulation by Rho GTPases, processes with demonstrated relevance for HSC emergence (Pereira et al., Dev Cell, 2016; Pereira et al., Cell Stem Cell, 2013). Gene ontology analysis of co-bound genes showed that cell motion and vasculature development were enriched terms. The ChIP-seq data were further interrogated for the regulatory interactions between the three hemogenic TFs. Both GATA2 and GFI1B bind their own loci at the initial stages of reprogramming suggesting auto-regulation as previously shown in hematopoietic progenitors. In addition, GATA2 binds to a CpG island in the FOS locus, and GFI1B binds to the GATA2 locus only in the presence of the other two TFs. Binding of GATA2 to the GFI1B locus was not detected, suggesting that this interaction may be established later in hematopoietic progenitors. To confirm physical interaction, Co-IP was performed 48 hours after expression in fibroblasts. This analysis demonstrated an interaction between GATA2 and FOS and between GATA2 and GFI1B. This suggests that the interplay between GATA2, FOS, and GFI1B plays an import role for hemogenic reprogramming.

Next, whether GATA2 and/or GFI1B engagement correlates with gene activation or silencing during human reprogramming was investigated. 1425 significantly changing genes (across the population mRNA-seq dataset from HDF-derived cells), bound by either GATA2 and/or GFI1B, were identified. Specifically, 1186 genes were bound by GATA2 and 182 were bound only by GFI1B. Fifty-seven differentially expressed genes were co-bound, targeting the cluster of genes highly expressed in fibroblasts and a second cluster of genes enriched only in CD34+CD49f+cells. This data suggests that GATA2 and GFI1B co-binding is in part involved both in the repression of fibroblast-associated genes and activation of hematopoietic-associated genes. To characterize the chromatin features associated with GATA2 and GFI1B engagement, previously published ChIP-seq datasets for H3K4me1, H3K4me3, H3K27ac, H3K27me3, H3K9me3 and H3K36me3 in HDFs were used. GATA2 and GFI1B bound sites in fibroblasts are enriched for marks associated with active promoters and enhancers such as H3K4me3, H3K27ac, and H3K4me1. This result is consistent with the DNase I accessibility in human dermal fibroblasts. GATA2 and GFI1B bind mostly to DNase I sensitive sites. These results demonstrate that GATA2 and GFI1B preferentially bind to accessible chromatin primarily in promoter and enhancer regions. The association between GATA2 and GFI1B binding and chromatin in fibroblasts was investigated using ChromHMM, a segmentation of the genome into 18 chromatin states based on the combinatorial patterns of chromatin marks. The results confirm the preference of GATA2 and GFI1B in active TSS, flanking upstream TSS and active enhancers. In addition, published data sets were analyzed for histones marks in K562 cells and GATA2, GFI1B, and FOS transcription factor occupancy in Hematopoietic Progenitor Cells (HPCs). In contrast to GATA2 and FOS, the inventors observed a distinct pattern for GFI1B that is strongly enriched in bivalent/poised TSS. This dramatic shift in GFI1B targeting suggests that the cooperative interaction between GATA2 and GFI1B may be specific for the earlier stages of hematopoietic reprogramming and EHT that is lost in downstream hematopoietic progenitors.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

1. A method for programming a human somatic cell into a hematopoietic stem cell, the method comprising introducing into the human somatic cell a combination of transcription factors, wherein the combination comprises GATA binding protein 2 (GATA2), growth factor independent 1B (GFI1B), growth factor independent 1 (GFI1), and FBJ osteosarcoma oncogene (FOS).

2. The method of claim 1, wherein the human somatic cell is selected from the group consisting of: fibroblasts, epithelial cells, bone marrow cells, differentiated hematopoietic cells, macrophages, and hematopoietic progenitor cells, and peripheral blood mononuclear cells.

3. The method of claim 1, wherein the step of introducing further comprises introducing the combination of transcription factors into the human somatic cell by viral transduction.

4. The method of claim 1, further comprising the step of screening the cell for expression of a hemogenic endothelial cell marker or a hematopoietic stem cell marker.

5. The method of claim 4, wherein the hemogenic endothelial cell marker or the hematopoietic stem cell marker is a marker selected from the group consisting of: CD31, CD34, CD38^{lo/-}, CD41, CD43, CD45, CD49f, Thy1/CD90, CD105, CD117/c-kit, CD133, CD143, CD150, CD201, Sca-1, Tie2, VE-Cadherin, KDR/FLK1, Flk-2/Flt3, and CXCR4.

6. The method of claim 4, wherein the hematopoietic stem cell marker is CD34 or CD49f.

7. The method of claim 1, further comprising the step of isolating the cell expressing the hematopoietic stem cell marker.

8. The method of claim 1, further comprising the step of co-culturing the hematopoietic stem cell with a stromal cell.

9. The method of claim 8, wherein the stromal cell is an AFT024 stromal cell.

10. An isolated hematopoietic stem cell obtained by the method of claim 1.

11. A composition comprising the isolated hematopoietic stem cell of claim 10 and a cryo-protectant.

12. Blood, cellular and acellular blood components, blood products or hematopoietic stem cells comprising the isolated hematopoietic cells of claim 10.

13. A method of engraftment or cell replacement for autologous or non-autologous transplantation in a subject in need thereof comprising transferring to the subject the isolated hematopoietic cells of claim 10.

14. A method for treating a subject who suffers from a condition or a disease that would benefit from hematopoietic stem cell transplantation, comprising administering to the subject a therapeutically effective amount of the isolated hematopoietic stem cells of claim 10, wherein the condition or disease is selected from the group consisting of cancer, a congenital disorder, and vascular disease.

15. A method for treating a subject who suffers from a condition or a disease that would benefit from hematopoietic stem cell transplantation, comprising administering to the subject a therapeutically effective amount of the isolated hematopoietic stem cells of claim 10, wherein the condition or disease is selected from the group consisting of multiple myeloma, leukemia, congenital neutropenia with defective stem cells, aplastic anemia, myelodysplastic syndrome, neuroblastoma, lymphoma, Ewing's Sarcoma, Desmoplastic small round cell tumor, chronic granulomatous disease, non-Hodgkin's lymphoma, Hodgkin's disease, acute myeloid leukemia, neuroblastoma, germ cell tumors, systemic lupus erythematosus (SLE), systemic sclerosis, amyloidosis, acute lymphoblastic leukemia, chronic myeloid leukemia, chronic lymphocytic leukemia, myeloproliferative disorders, myelodysplastic syndromes, pure red cell aplasia, paroxysmal nocturnal hemoglobinuria, Fanconi anemia, Thalassemia major, sickle cell anemia, severe combined immunodeficiency (SCID), Wiskott-Aldrich syndrome, hemophagocytic lymphohistiocytosis (HLH), mucopolysaccharidosis, Gaucher disease, metachromatic leukodystrophy, adrenoleukodystrophy, vascular disease, ischemia, and atherosclerosis.

16. A method for treating a subject who suffers from a condition or a disease that would benefit from hematopoietic stem cell transplantation, comprising administering to the subject a therapeutically effective amount of the isolated hematopoietic stem cells of claim 10, or committed or differentiated progeny thereof, wherein the condition or disease is selected from the group consisting of: cancer, multiple myeloma, leukemia, congenital neutropenia with defective stem cells, aplastic anemia, myelodysplastic syndrome, neuroblastoma, lymphoma, Ewing's Sarcoma, Desmoplastic small round cell tumor, chronic granulomatous disease, non-Hodgkin's lymphoma, Hodgkin's disease, acute myeloid leukemia, neuroblastoma, germ cell tumors, systemic lupus erythematosus (SLE), systemic sclerosis, amyloidosis, acute lymphoblastic leukemia, chronic myeloid leukemia, chronic lymphocytic leukemia, myeloproliferative disorders, myelodysplastic syndromes, pure red cell aplasia, paroxysmal nocturnal hemoglobinuria, Fanconi anemia, Thalassemia major, sickle cell anemia, severe combined immunodeficiency (SCID), Wiskott-Aldrich syndrome, hemophagocytic lymphohistiocytosis (HLH), mucopolysaccharidosis, Gaucher disease, metachromatic leukodystrophy, adrenoleukodystrophy, vascular disease, ischemia, and atherosclerosis.

17. The method of claim 14, wherein the isolated hematopoietic stem cell is autologous to the subject in need thereof

18. A method for testing the toxicity of a compound on a population of hematopoietic stem cells, the method comprising:
administering the compound to a population of the isolated hematopoietic stem cells of claim 10; and
comparing the response of the isolated hematopoietic stem cells exposed to the compound to the isolated hematopoietic stem cells not exposed to the compound.
